SEQUENCE LISTING (3 sequences)

SEQ ID NO: 1 - protein, 325 aa, Homo sapiens
MVCGSPGGMLLLRAGLLALAALCLLRVPGARAAACEPVRIPLCKSLPWNMTKMPNHLHHSTQANAILAIEQFEGLLGTHCSPDLLFFLCAMYAPICTIDFQHEPIKPCKSVCERARQGCEPILIKYRHSWPENLACEELPVYDRGVCISPEAIVTADGADFPMDSSNGNCRGASSERCKCKPIRATQKTYFRNNYNYVIRAKVKEIKTKCHDVTAVVEVKEILKSSLVNIPRDTVNLYTSSGCLCPPLNVNEEYIIMGYEDEERSRLLLVEGSIAEKWKDRLGKKVKRWDMKLRHLGLSKSDSSNSDSTQSQKSGRNSNPRQARN

SEQ ID NO: 2 - protein, 125 aa, Homo sapiens
AACEPVRIPLCKSLPWNMTKMPNHLHHSTQANAILAIEQFEGLLGTHCSPDLLFFLCAMYAPICTIDFQHEPIKPCKSVCERARQGCEPILIKYRHSWPENLACEELPVYDRGVCISPEAIVTAD

SEQ ID NO: 3 - DNA, 2058 bp, Homo sapiens
gttgggaaagagcagcctgggcggcaggggcggtggctggagctcggtaaagctcgtgggaccccattgggggaatttgatccaaggaagcggtgattgccgggggaggagaagctcccagatccttgtgtccacttgcagcgggggaggcggagacggcggagcgggccttttggcgtccactgcgcggctgcaccctgccccatcctgccgggatcatggtctgcggcagcccgggagggatgctgctgctgcgggccgggctgcttgccctggctgctctctgcctgctccgggtgcccggggctcgggctgcagcctgtgagcccgtccgcatccccctgtgcaagtccctgccctggaacatgactaagatgcccaaccacctgcaccacagcactcaggccaacgccatcctggccatcgagcagttcgaaggtctgctgggcacccactgcagccccgatctgctcttcttcctctgtgccatgtacgcgcccatctgcaccattgacttccagcacgagcccatcaagccctgtaagtctgtgtgcgagcgggcccggcagggctgtgagcccatactcatcaagtaccgccactcgtggccggagaacctggcctgcgaggagctgccagtgtacgacaggggcgtgtgcatctctcccgaggccatcgttactgcggacggagctgattttcctatggattctagtaacggaaactgtagaggggcaagcagtgaacgctgtaaatgtaagcctattagagctacacagaagacctatttccggaacaattacaactatgtcattcgggctaaagttaaagagataaagactaagtgccatgatgtgactgcagtagtggaggtgaaggagattctaaagtcctctctggtaaacattccacgggacactgtcaacctctataccagctctggctgcctctgccctccacttaatgttaatgaggaatatatcatcatgggctatgaagatgaggaacgttccagattactcttggtggaaggctctatagctgagaagtggaaggatcgactcggtaaaaaagttaagcgctgggatatgaagcttcgtcatcttggactcagtaaaagtgattctagcaatagtgattccactcagagtcagaagtctggcaggaactcgaacccccggcaagcacgcaactaaatcccgaaatacaaaaagtaacacagtggacttcctattaagacttacttgcattgctggactagcaaaggaaaattgcactattgcacatcatattctattgtttactataaaaatcatgtgataactgattattacttctgtttctcttttggtttctgcttctctcttctctcaacccctttgtaatggtttgggggcagactcttaagtatattgtgagttttctatttcactaatcatgagaaaaactgttcttttgcaataataataaattaaacatgctgttaccagagcctctttgctggagtctccagatgttaatttactttctgcaccccaattgggaatgcaatattggatgaaaagagaggtttctggtattcacagaaagctagatatgccttaaaacatactctgccgatctaattacagccttatttttgtatgccttttgggcattctcctcatgcttagaaagttccaaatgtttataaaggtaaaatggcagtttgaagtcaaatgtcacataggcaaagcaatcaagcaccaggaagtgtttatgaggaaacaacacccaagatgaattatttttgagactgtcaggaagtaaaataaataggagcttaagaaagaacattttgcctgattgagaagcacaactgaaaccagtagccgctggggtgttaatggtagcattcttcttttggcaatacatttgatttgttcatgaatatattaatcagcattagagaaatgaattataactagacatctgctgttatcaccatagttttgtttaatttgcttccttttaaataaacccattggtgaaagtcccaaaaaaaaaaaaaaaaaaaaa

(19) United States
(12) Patent Application Publication
(10) Pub. No.: US 2010/0261659 A1
(43) Pub. Date: Oct. 14, 2010

(54) SECRETED FRIZZLED RELATED PROTEIN 3 FOR USE IN THE INHIBITION OF SCARRING

(75) Inventors: Mark W. J. Ferguson, Manchester (GB); Hugh Laverty, Manchester (GB); Nicholas Occleston, Manchester (GB); Sharon O'Kane, Manchester (GB); Kerry Nield, Manchester (GB)

Correspondence Address: FOLEY AND LARDNER LLP;SUITE 500, 3000 K STREET NW, WASHINGTON, DC, 20007, US

(73) Assignee: RENOVO LIMITED

(21) Appl. No.: 12/596,332

(22) PCT Filed: Apr. 17, 2008
(86) PCT No.: PCT/GB08/01345

§ 371 Date: Oct. 16, 2009

(30) Foreign Application Priority Data
Apr. 17, 2007 (GB) 0707348.9

(51) Int. Cl. A61K 38/17; A61P 17/02

(52) U.S. Cl. 514/20.6

(57) ABSTRACT

Provided is secreted Frizzled Related Protein 3 (sFRP3), or a therapeutically effective fragment or derivative thereof, for use as a medicament for the prevention, reduction or inhibition of scarring. The scarring may be associated with the healing of a wound, or with a fibrotic disorder. The scarring may be associated with surgical wounds. The scarring may be scarring of the skin. The medicament may be a topical medicament, and may be suitable for local injection. Also provided is a method of preventing, reducing or inhibiting scarring, the method comprising administering a therapeutically effective amount of sFRP3, or a therapeutically effective fragment or derivative thereof, to a patient in need of such prevention, reduction or inhibition.

The present invention relates to medicaments for the prevention, reduction or inhibition of scarring. The invention also provides methods for the prevention, reduction or inhibition of scarring. The medicaments or methods of the invention may be used for the prevention, reduction or inhibition of scarring associated with wounds or of scarring associated with fibrotic disorders.

Clinical approaches to wound management will generally depend on the desired outcome. This outcome may, for example, be considered with reference to the degree of scarring occurring, or with reference to the speed at which a wound heals. In management of some wounds control of the degree of scarring that occurs is of primary importance, while increasing the speed of wound healing is of much lesser importance. In management of other wounds increasing the speed of wound healing is of primary importance, while controlling the degree of scarring occurring is of much lesser importance. The present invention is preferably applicable to the management of wounds in which the primary clinical concern relates to the degree of scarring arising as a result of healing.

Many different processes are at work during the scarring response, and much research has been conducted into discovering what mediates these processes, and how they interact with each other to produce the final outcome.

The scarring response is common throughout all adult mammals. Scarring may result from healing of a wound, or through the deposition of scar tissue associated with fibrotic disorders. The scarring response is conserved between the majority of tissue types and in each case leads to the same result, formation of fibrotic tissue termed a “scar”. A scar may be defined as “fibrous connective tissue that forms at the site of injury or disease in any tissue of the body”.

In the case of a scar that results from healing of a wound, the scar constitutes the structure produced as a result of the reparative response. This reparative process has arisen as the evolutionary solution to the biological imperative to prevent the death of a wounded animal. In order to overcome the risk of mortality due to infection or blood loss, the body reacts rapidly to repair the damaged area, rather than attempt to regenerate the damaged tissue. Since the damaged tissue is not regenerated to attain the same tissue architecture present before wounding, a scar may be identified by virtue of its abnormal morphology as compared to unwounded tissue.

Although scarring may most frequently occur on healing of a wound, similar disturbances of the extracellular matrix may also give rise to scarring associated with a number of medical conditions known as fibrotic disorders. In these disorders excessive fibrosis leads to pathological derangement and malfunctioning of tissue. Scars associated with fibrotic disorders are characterised by the accumulation of fibrous tissue in an abnormal fashion within the diseased area. Accumulation of such fibrous tissues may result from a variety of disease processes, all of which are capable of leading to the production of a scar.

Fibrotic disorders are usually chronic. Examples of fibrotic disorders include cirrhosis of the liver, liver fibrosis, glomerulonephritis, pulmonary fibrosis, chronic obstructive pulmonary disease, scleroderma, myocardial fibrosis, fibrosis following myocardial infarction, proliferative vitreoretinopathy (PVR), arthritis and adhesions e.g. in the digestive tract, abdomen, pelvis, spine. If left untreated, the pathological effects of scarring associated with fibrotic disorders may lead to organ failure, and ultimately to death.

The biological and pathological processes underlying the development of scars associated with fibrotic disorders are sufficiently similar to those involved in the formation of scars resulting from healing of a wound, that those compounds that may be used to prevent, reduce or inhibit scarring associated with one form will generally be similarly effective in the other form of scarring.

Scars, whether produced as a result of wounds or of fibrotic disorders, are composed of connective tissue. In the case of wounds this material is deposited during the healing process, whereas in fibrotic disorders it occurs as a result of the disease process. A scar may comprise connective tissue that has an abnormal organisation, as is frequently observed in scars of the skin. Alternatively or additionally, a scar may comprise connective tissue that is present in an abnormally increased amount. Most scars consist of both abnormally organised and excess connective tissue, as described further below.

The abnormal structure of scars may be observed with reference to both their internal structure (which may be determined by means of microscopic analysis) and their external appearance (which may be assessed macroscopically).

In connective tissues, such as the skin, extracellular matrix (ECM) molecules comprise the major structural component of both “normal” (unwounded) and scarred tissues. In normal skin these molecules form fibres which, when viewed microscopically, have a characteristic random arrangement that is commonly referred to as “basket-weave”. This basket-weave arrangement is disrupted in scars. Fibres in scars exhibit a marked degree of alignment with each other as compared to the random arrangement of fibres in normal skin. In general the fibres observed within scars are also of smaller diameter than those seen in normal skin. Both the size and arrangement of ECM may contribute to the scars altered mechanical properties, most notably increased stiffness, when compared with normal skin.

Viewed macroscopically, scars may be depressed below the surface of the surrounding tissue, or elevated above the surface of their undamaged surroundings. Scars may be relatively darker coloured than normal tissue (hyperpigmentation) or may have a paler colour (hypopigmentation) compared to their surroundings. In the case of scars of the skin, either hyperpigmented or hypopigmented scars constitute a readily apparent cosmetic defect. It is also known that scars of the skin may be redder than unwounded skin, causing them to be noticeable and cosmetically unacceptable. It has been shown that the cosmetic appearance of a scar is one of the major factors contributing to the psychological impact of scars upon the sufferer, and that these effects can remain long after the cause of the scar, be it either a wound or a fibrotic disorder, has passed.

In addition to their psychological effects, scars may also have deleterious physical effects upon the sufferer. These effects typically arise as a result of the mechanical differences between scars and normal tissue. The abnormal structure and composition of scars mean that they are typically less flexible than their normal tissue counterpart. As a result scars may be responsible for impairment of normal function (such as in the case of scars covering joints which may restrict the possible range of movement) and may retard normal growth if present from an early age.

Scars occur at many body sites, and the effects of scarring at these sites will generally be related to loss or disruption of function in the scarred area. Some of the disadvantages associated with scarring of the skin have been discussed above. Scarring in the eye (whether as a result of accidental injury, surgical intervention, or a fibrotic disorder) can impair vision and even lead to blindness. Scarring of the internal organs may lead to the formation of strictures and adhesions that significantly or totally impair function of the organ in question. Scarring of tendons and ligaments may cause lasting damage to these organs, and thereby reduce the motility or function of associated joints. Scarring associated with blood vessels, and particularly the valves of the heart, may occur after injury or surgery. Scarring of blood vessels may lead to restenosis, which causes a narrowing of the blood vessel and thus reduces the flow of blood through the scarred area. Scarring in the central and peripheral nervous system may prevent transmission along the nerve and may prevent or reduce reconnection of damaged nerve tissue, and/or functional neuronal transmission.

The effects outlined above may all arise as a result of the normal progression of the wound healing response (in the case of scars that result from healing of a wound). There are, however, many ways in which the scarring response may be abnormally altered; and these are frequently associated with even more damaging effects resulting from the production of abnormal excessive scarring (commonly referred to as pathological scarring). The most frequent and important classes of pathological scarring include hypertrophic scarring, keloid scarring and pterygium, and these are discussed elsewhere in the specification.

Whilst much of the present specification concentrates primarily on the effects of scarring in man (whether scarring that results from healing of a wound, or scarring associated with fibrotic disorders), it will be appreciated that many aspects of the scarring response are conserved between most species of animals. Thus, the problems outlined above are also applicable to non-human animals, and particularly veterinary or domestic animals (e.g. horses, cattle, dogs, cats etc). By way of example, it is well known that adhesions resulting from the inappropriate healing of abdominal wounds constitute a major reason for the veterinary destruction of horses (particularly race horses). Similarly the tendons and ligaments of domestic or veterinary animals are also frequently subject to injury, and healing of these injuries may also lead to scarring associated with increased animal mortality.

Although the ill effects of scarring (either resulting from normal or aberrant wound healing, or associated with fibrotic disorders) are well known there remains a lack of effective therapies able to reduce these effects. In the light of this absence it must be recognised that there exists a strongly felt need to provide medicaments and treatments that are able to prevent, reduce or inhibit scar formation, whether resulting from healing of a wound, or associated with fibrotic disorders.

The secreted Frizzled-related proteins (sFRPs) are a group of secreted glycoproteins which structurally resemble the Frizzled family of proteins (which are receptors for WNT signalling proteins). sFRPs possess a cysteine-rich domain (CRD) homologous to the CRD of Frizzled proteins but lack the transmembrane and cytosolic domains of the full length Frizzled proteins. The CRDs of sFRPs, which share 30-50% sequence similarity with those of Frizzled proteins, include ten conserved cysteine residues which are shared with the Frizzled CRD domain, in addition to further conserved residues such as a proline located four residues C-terminal to cysteine-9. The domain spans 120-125 amino acids near the N-terminus of the protein (Melkonyan et al., 1997; Jones and Jomary 2002; Dann et al., 2001).

The majority of sFRPs, though not all, are able to antagonise WNT signalling. The mechanism of this antagonism has not been fully elucidated, but it has been suggested that it is brought about by a direct interaction between WNT and either the CRD, or the C-terminal domain lying outside the CRD. It is believed that sFRPs may block WNT signalling either by interacting with WNT proteins, and thus preventing binding of WNTs to Frizzled receptors, or by forming non-functional complexes with the Frizzled receptors themselves.

Secreted Frizzled Related Protein 3 (sFRP3) is a member of the sFRP family, and is also known as Frizzled-related protein 1, FrzB-1, Frezzled, and Fritz. The amino acid sequence of the human sFRP3 protein is shown in Sequence ID No. 1 (this sequence had previously been deposited as Accession Number: Q92765).

The N-terminal CRD domain of sFRP-3 has been shown to be able to bind and inhibit, the activity of WNT1 and WNT8. The amino acid sequence of the CRD of human sFRP3, including conserved cysteine residues, is shown in Sequence ID No. 2. sFRP3 has also been shown to bind WNT5A in immuno-precipitation experiments, although this interaction is not sufficient to block WNT5A activity. DNA encoding human sFRP3 is shown in Sequence ID No. 3 (this sequence has previously been deposited as Accession Number NM_{—}001463).

It is an aim of certain aspects of the present invention to provide medicaments suitable for the prevention and/or reduction and/or inhibition of scarring. It is an aim of further aspects of the present invention to provide methods of treatment suitable for use in the prevention, and/or reduction, and/or inhibition of scarring. It is an aim of certain embodiments of the invention to provide medicaments suitable for the prevention and/or treatment of scarring that results from healing of a wound. It is an aim of certain embodiments of the invention to provide medicaments suitable for the prevention and/or treatment of scarring associated with fibrotic disorders. It is an aim of certain embodiments of the invention to provide methods of treatment suitable for use in the prevention and/or treatment of scarring that results from healing of a wound. It is an aim of further embodiments of the invention to provide methods of treatment suitable for use in the prevention and/or treatment of scarring associated with fibrotic disorders. The medicaments and/or methods of the invention may constitute alternatives to those provided by the prior art. However, it is preferred that medicaments and/or methods of treatment provided by the invention may constitute improvements over the prior art.

According to a first aspect of the present invention there is provided the use of secreted Frizzled Related Protein 3 (sFRP3), or a therapeutically effective fragment or derivative thereof, in the manufacture of a medicament for the prevention, reduction or inhibition of scarring. This first aspect of the invention also provides sFRP3, or a therapeutically effective fragment or derivative thereof, for use as a medicament for the prevention, reduction or inhibition of scarring. The medicament may be a topical medicament for application at a site where scarring is to be prevented, reduced or inhibited. The medicament may preferably be for use at a wound, or at a site where a wound is to be formed. The medicament may be suitable for localised injection, such as intradermal injection.

In a second aspect of the invention there is provided a method of preventing, reducing or inhibiting scarring, the method comprising administering a therapeutically effective amount of sFRP3, or a therapeutically effective fragment or derivative thereof, to a patient in need of such prevention, reduction or inhibition. The sFRP3, or therapeutically effective fragment or derivative thereof, may preferably be administered to the site where scarring is to be prevented, reduced or inhibited. The site may preferably be a wound, or a site where a wound is to be formed.

It may be preferred that the medicaments or methods of the invention utilise sFRP3 itself. It will be appreciated that the sFRP3 to be used will generally be human sFRP3, as set out in Sequence ID No. 1.

The scarring, prevention, reduction or inhibition of which is to be achieved by the medicaments or methods of the invention, may be scarring that results from healing of a wound, or, additionally or alternatively, may be scarring associated with a fibrotic disorder. It may generally be preferred that scarring to be prevented, reduced or inhibited is scarring that results from the healing of a wound.

The inventors believe that the prevention, reduction or inhibition of scarring using sFRP3, or therapeutically effective fragments or derivatives thereof, can be effected at any body site and in any tissue or organ. However, the skin represents a preferred organ in which scarring may be prevented, reduced or inhibited utilising the medicaments or methods of the invention. Such scarring of the skin may result from healing of a skin wound and/or may be associated with a fibrotic disorder involving the skin.

Scarring resulting from the healing of skin wounds represents a form of scarring that may particularly benefit from prevention, reduction or treatment in accordance with the present invention, and with the medicaments or methods of the present invention. Accordingly, it will also be recognised that skin wounds, or sites where skin wounds are to be formed, may beneficially be treated using the medicaments or methods of the invention.

The present invention is based on the inventors' new and surprising finding that sFRP3, or therapeutically effective fragments or derivatives thereof, may be used in the prevention, reduction or inhibition of scarring. The inventors have found that this anti-scarring effect of sFRP3 is exerted by all doses of this molecule so far investigated.

Without wishing to be bound by any hypothesis, the inventors believe that the prevention, reduction or inhibition of scarring observed arises as a result of the ability of sFRP3 to antagonise WNT signalling. This antagonism may occur through the action of the CRD of sFRP3, or through the action of other regions of the C-terminal domain. There are no previous reports that would lead the skilled person to believe that sFRP3, or its fragments or derivatives, may be used to effectively prevent, reduce or inhibit scarring.

The finding that sFRP3, or fragments or derivatives thereof, may be used to prevent, reduce or inhibit scarring provides the foundation for new medicaments and methods that may be used in the treatment, management or improvement of scarring. Furthermore, the inventors' finding that sFRP3, or its fragments or derivatives, may be used in the prevention, reduction or inhibition of scarring offers the prospect that improved medicaments and methods may be made available for the treatment or management of scarring.

sFRP3, or a therapeutically effective fragment or derivative thereof, may preferably be administered to a site that may be associated with scarring (for the present purposes a site where scarring has already occurred, is occurring, or may be expected to occur). For example, sFRP3, or therapeutically effective fragments or derivatives thereof, may be provided to a patient's wound that would otherwise be likely to give rise to a scar, or may be provided to a site where an increased likelihood of fibrosis has been identified.

sFRP3, or a therapeutically effective fragment or derivative thereof, may be administered to an existing scar to prevent the further progression of scarring. Administration of sFRP3, or therapeutically effective fragments or derivatives thereof, to an existing scar may also reduce the level of scarring associated with the existing scar. It will thus be appreciated that sFRP3, or a therapeutically effective fragment or derivative thereof, may be administered to a site of a fibrotic disorder in order to prevent further scarring, and/or to reduce scarring that has already occurred in association with the fibrotic disorder. Preferred routes of administration that may be used in accordance with all of the embodiments considered above include topical administration, and particularly topical injection of suitable active agents.

Various terms that are used in the present disclosure to describe the invention will now be explained further. The definitions and guidance provided below may be expanded on elsewhere in the specification as appropriate, and as the context requires.

“Therapeutically Effective Fragments or Derivatives of sFRP3”

For the purpose of the present disclosure, “therapeutically effective fragments or derivatives of sFRP3” should be taken (except for where the context requires otherwise) to encompass any fragment or derivative of sFRP3 that is capable of inhibiting scarring. Preferred means by which such inhibition of scarring may be assessed (and quantified if required) are considered elsewhere in the specification.

Except for where the context requires otherwise, it should be considered that therapeutically effective derivatives suitable for use in the medicaments or methods of the invention may be derived either from sFRP3 itself, or from therapeutically effective fragments of sFRP3. Preferred fragments or derivatives of sFRP3 for use in the medicaments and methods of the invention may be those based on human sFRP3, the amino acid sequence of which is shown in Sequence ID No. 1. The biological activity of sFRP3 has frequently been ascribed to the CRD of sFRP3 and/or to areas of the C-terminal domain of sFRP3. Accordingly, preferred therapeutically effective fragments of sFRP3 for use in medicaments or methods of the invention may be fragments comprising all, or part, of the CRD (as shown in Sequence ID No. 2), or of the C-terminal domain of sFRP3.

A therapeutically effective fragment or derivative of sFRP3 may be a fragment or derivative that is effective to inhibit scarring by at least 10% compared to a suitable control. Preferably a therapeutically effective fragment or derivative of sFRP3 may be capable of inhibiting scarring by at least 20%, more preferably at least 50%, even more preferably at least 75% and yet more preferably by at least 90% compared to a suitable control. A most preferred therapeutically effective fragment or derivative of sFRP3 may be capable of inhibiting scarring by 100% as compared to a suitable control.

In particular, therapeutically effective fragments or derivatives of sFRP3 suitable for use in the medicaments or methods of the invention may be those able to alter the amount and/or orientation of extracellular matrix components (such as collagen) present in a treated scar and thereby inhibit scarring. A therapeutically effective fragment or derivative of sFRP3 suitable for use in the medicaments or methods of the invention may be one that is able to give rise to a treated scar in which the ECM architecture is like that of unwounded tissue.

Preferably a therapeutically effective fragment or derivative of sFRP3 may be one that is capable of inhibiting scarring at a site to which the fragment or derivative of sFRP3 is administered. Such a site may be a wound, or scar resulting from the healing of a wound. Alternatively or additionally, such a site may be a site of a fibrotic disorder.

Suitable therapeutically effective amounts of sFRP3, as well as suitable therapeutically effective fragments or derivatives of sFRP3, are considered elsewhere in the specification.

“Therapeutically Effective Fragments”

Therapeutically effective fragments of sFRP3 suitable for use in accordance with the present invention may comprise 10 or more amino acid residues from Sequence ID No. 1, preferably up to 100 amino acid residues, more preferably up to 200 amino acid residues, and even more preferably up to 300 amino acid residues. Fragments suitable for use in the medicaments and methods of the present invention include those comprising up to 324 amino acids residues of Sequence ID No. 1.

Preferred therapeutically effective fragments of sFRP3 suitable for use in the medicaments or methods of the invention include those comprising some, or all, of the CRD domain of sFRP3 (shown in Sequence ID No. 2), and/or those comprising some, or all, of the C-terminal domain of sFRP3.

Therapeutically effective fragments of sFRP3 suitable for use in accordance with the present invention may comprise up to 10 contiguous amino acid residues from Sequence ID No. 2, preferably up to 50 contiguous amino acid residues, more preferably up to 100 contiguous amino acid residues, and even more preferably up to 124 contiguous amino acid residues from Sequence ID No. 2.

Preferred fragments may include amino acid residues involved in binding of sFRP3 to its biological targets, including WNTs such as WNT1 and WNT8. Preferred therapeutically effective fragments or derivatives of sFRP3 will be those that incorporate a WNT-binding region of sFRP3 (either in whole or in part), such as the CRD of sFRP3, or C-terminal domain of sFRP3. Preferred fragments of sFRP3 for use in the medicaments or methods of the invention may include those comprising at least five of the conserved cysteine residues in the CRD of sFRP3, and preferably may comprise at least six, seven, eight, nine or all ten of these residues.

It will be appreciated that it is the three dimensional structure of sFRP3 that is important in considering its binding to biological targets, and that accordingly suitable fragments may be selected based upon their ability to assume the requisite three dimensional conformation necessary for target binding.

In a further aspect, the invention provides the use of an agent comprising a therapeutically effective portion of the CRD of sFRP3 in the manufacture of a medicament for the prevention, reduction or inhibition of scarring. The therapeutically effective portion of the CRD of sFRP3 may preferably be the entire CRD, as shown in Sequence ID No. 2. Such medicaments may be used to prevent, reduce or inhibit scarring of the various types described elsewhere in the specification (whether scarring as a result of healing of wounds, or as a result of fibrotic disorders). Medicaments according to this embodiment of the invention may be formulated and/or administered in accordance with information provided throughout the present disclosure.

“Therapeutically Effective Derivatives”

Although peptides comprising all or part of sFRP3 (as defined by Sequence ID No. 1) represent preferred agents for use in accordance with the present invention, it will be recognised that there are contexts in which the sensitivity of peptides to degradation may be disadvantageous. There are many known techniques by which peptide derivatives may be produced that have greater resistance to degradation than do the original peptides from which they are derived. Such derivatives may represent preferred active agents suitable for use in accordance with the invention.

Preferred therapeutically effective derivatives of sFRP3 for use in the medicaments or methods of the invention may include derivatives corresponding to all or part of the CRD of sFRP3, or to the C-terminal domain of sFRP3, as considered with reference to therapeutically effective fragments above. Therapeutically effective derivatives of such fragments represent preferred derivatives in accordance with the present invention. The medicaments or methods of the invention may make use of therapeutically effective peptides derivable from the CRD of sFRP3.

Suitable therapeutically effective derivatives of sFRP3 for use in the medicaments or methods of the invention may be selected from the group consisting of: therapeutically effective derivatives based on the pharmacophore of sFRP3; therapeutically effective peptoid derivatives of sFRP3 or its fragments; therapeutically effective D-amino acid derivatives of sFRP3 or its fragments; therapeutically effective peptidomimetics based on sFRP3 or its fragments; therapeutically effective peptide analogues of sFRP3 or its fragments; therapeutically effective pseudopeptides based on sFRP3 or its fragments; therapeutically effective retro-inverso peptides based on sFRP3 or its fragments; therapeutically effective depsipeptide derivatives based on sFRP3 or its fragments; therapeutically effective β-peptide derivatives based on sFRP3 or its fragments; therapeutically effective small molecule mimics of sFRP3 or its fragments and therapeutically effective retropeptoid derivatives based on sFRP3 or its fragments.

Peptoid derivatives may be expected to have greater resistance to degradation than do peptide agents of the invention, whilst retaining the same ability to inhibit scarring. Suitable peptoid derivatives may be readily designed from knowledge of sFRP3's sequence and structure (and in particular the sequence and structure of the CRD and/or C-terminal domain). Commercially available software and well-established protocols may be used to develop peptoid derivatives suitable for use in accordance with the invention. It will be appreciated that the therapeutic effectiveness of peptoid and other derivatives may be investigated using the same techniques that allow assessment of therapeutic effectiveness of peptide fragments.

Retropeptoids (based on sFRP3 or its therapeutically effective fragments) in which all amino acids are replaced by peptoid residues in reversed order may also be used in the medicaments or methods of the invention to inhibit scarring. A retropeptoid may be expected to bind to its binding partner in the opposite direction to the naturally occurring peptide or a peptoid-peptide hybrid containing one peptoid residue.

D-amino acid forms of sFRP3 or its therapeutically effective fragments also confer the requisite ability to inhibit scarring. In the case of D-amino acid forms, the order of the amino acid residues comprising the derivative is reversed as compared to those in the original peptide. The preparation of derivatives using D-amino acids rather than L-amino acids greatly decreases any unwanted breakdown of such an agent by normal metabolic processes, decreasing the amounts of agent which need to be administered, along with the frequency of its administration.

It will be appreciated that derivatives suitable for use in the medicaments and methods of the invention clearly include both those derived from full length sFRP3 and those derived from therapeutically effective fragments of sFRP3.

Derivatives of sFRP3 suitable for use in the medicaments or methods of the invention also include peptide derivatives capable of inhibiting scarring. Such peptide derivatives may be based on sFRP3, or on fragments thereof, but may comprise alterations or substitutions of the naturally occurring amino acid sequence. It will be appreciated that amino acid residues involved in the binding of sFRP3 to WNTs may be retained in peptide derivatives for use in accordance with the invention, but that amino acid residues that are not involved in binding to WNTs may be substituted without adversely effecting the activity of such derivatives.

The group of therapeutically effective derivatives of sFRP3 also encompasses therapeutically effective small molecule mimics of sFRP3, or its fragments. For the purposes of the present disclosure, small molecule mimics of sFRP3 encompass any small molecule chemical entities that are able to mimic sFRP3 activity, and thereby inhibit scarring, for example by inhibiting WNT signalling. It may be preferred that small molecule mimics of sFRP3 are organic molecules with a weight less than 330 kDa, and preferably with a weight less than 1 kDa. Suitable small molecule mimics of sFRP3 that may be used in the medicaments or methods of the invention include those that are able to inhibit the scarring of a wound to which they are provided.

A therapeutically effective derivative of sFRP3 suitable for use in accordance with the present invention may share at least 10% homology with Sequence ID No. 1, preferably at least 25% homology, more preferably at least 50% homology, and even more preferably at least 75% homology. Particularly preferred derivatives may share at least 80%, 85%, 90%, 95% or greater homology with Sequence ID No. 1.

Therapeutically effective derivatives of sFRP3 suitable for use in accordance with the present invention may share at least 10% identity with Sequence ID No. 1, preferably at least 25% identity, more preferably at least 50% identity, and even more preferably at least 75% identity. Particularly preferred derivatives may share at least 80%, 85%, 90%, 95% or greater identity with Sequence ID No. 1.

“Therapeutically Effective Amounts”

A therapeutically effective amount of sFRP3, or a fragment or derivative thereof, is any amount of sFRP3, or a therapeutically effective fragment or derivative thereof, which is able to prevent, reduce or inhibit scarring. Such scarring may be associated with a wound or a fibrotic disorder.

A therapeutically effective amount of sFRP3, or a fragment or derivative thereof, is preferably an amount of sFRP3, or a fragment or derivative thereof, which is able to inhibit scarring of a wound (or site at which a wound is to be formed) or a fibrotic disorder (or site at which a fibrotic disorder will occur) to which the sFRP3, or fragment or derivative, is administered.

A therapeutically effective amount of a medicament of the invention is any amount of a medicament of the invention that is able to inhibit scarring. This inhibition of scarring may preferably be achieved at a site to which the medicament of the invention is administered.

A therapeutically effective amount of fragment or derivative of sFRP3, or of a medicament of the invention, may preferably be an mount of fragment or derivative that is effective to inhibit scarring by at least 10% compared to a relevant control. Preferably a therapeutically effective amount of sFRP3, or a fragment or derivative of sFRP3, or of a medicament of the invention, may be capable of inhibiting scarring by at least 20%, more preferably at least 50%, even more preferably at least 75% and yet more preferably of inhibiting scarring by at least 90% compared to a relevant control. A most preferred therapeutically effective amount of sFRP3, or a fragment or derivative of sFRP3, or a medicament of the invention, may be capable of inhibiting scarring by 100% as compared to a relevant control.

The selection of a suitable control will be apparent to one skilled in the art, but by way of guidance, in the event that it is wished to assess inhibition of scarring on healing of treated wounds, a suitable control may comprise an untreated or control treated wound. In the event that it is wished to assess inhibition of scarring achieved by provision of sFRP3, or a therapeutically effective fragment or derivative thereof, to an existing scar, an untreated scar may constitute a suitable control.

Thus a therapeutically effective amount of sFRP3, or a therapeutically effective fragment or derivative of sFRP3, or of a medicament of the invention, may be an amount that is effective to reduce scarring occurring on healing of a treated wound by at least 10% compared to scarring occurring on healing of an untreated or control wound. “Treated wounds” and “untreated wounds” or “control wounds” are defined elsewhere in the specification. Preferably a therapeutically effective amount may be capable of causing a 20% inhibition of scarring, more preferably at least a 50% inhibition, even more preferably at least a 75% inhibition and most preferably at least a 90% inhibition of the scarring occurring on healing of a treated wound as compared to scarring occurring on healing of an untreated or control wound.

In the case of scarring that may otherwise be associated with a fibrotic disorder, a therapeutically effective amount of sFRP3, or a therapeutically effective fragment or derivative of sFRP3, or of a medicament of the invention, may be an amount that is effective to reduce scarring of a treated site of fibrosis by at least 10% compared to the amount scarring that would otherwise be present at a comparable untreated site of fibrosis. A “treated site of fibrosis” and “untreated site of fibrosis” are defined further elsewhere in the specification. Preferably a therapeutically effective amount may be capable of achieving at least a 20% reduction in scarring, more preferably at least 50%, even more preferably at least 75% and most preferably at least a 90% reduction in scarring compared to scarring present at a comparable untreated site of fibrosis.

Suitable experimental or clinical models for the assessment of scarring (and thus of inhibition of scarring) will be well known to those skilled in the art. Suitable examples are set out elsewhere in the present specification.

The skilled person will appreciate that a fragment or derivative of sFRP3 that has little inherent therapeutic activity will still be therapeutically effective if administered in a quantity that provides a therapeutically effective amount.

A therapeutically effective amount of sFRP3, or a therapeutically effective fragment or derivative thereof, may preferably be an amount able to therapeutically alter the abundance and/or orientation of ECM components (such as collagen) in a treated scar.

The inventors have found that, in addition to being able to inhibit scar formation, sFRP3, or its therapeutically effective fragments or derivatives, is also able to accelerate the healing of wounds. Accordingly, a preferred therapeutically effective amount of sFRP3, or its fragments or derivatives, may be an amount that is able to inhibit scarring, and also to accelerate wound healing.

A medicament of the invention should provide a therapeutically effective amount of sFRP3, or a therapeutically effective fragment or derivative thereof. Preferably a medicament of the invention may be provided in the form of one or more dosage units. Each dosage unit may comprise a therapeutically effective amount of sFRP3, or a therapeutically effective fragment or derivative thereof, or a known fraction or multiple of such a therapeutically effective amount.

The inventors have found that sFRP3, or its therapeutically effective fragments or derivatives, are able to inhibit scarring at all doses investigated thus far.

The inventors believe that the provision of between approximately 0.1 ng and 1500 ng of sFRP3 per centimetre of wound or fibrosis (in a single incidence of treatment) may constitute a therapeutically effective amount in accordance with the present invention. Preferably a therapeutically effective amount of sFRP3 may be between about 1 ng and 1000 ng per centimetre of wound or fibrosis; more preferably between about 10 ng and 1000 ng; more preferably still between about 10 ng and 100 ng and most preferably about 10 ng of sFRP3 per centimetre of wound or fibrosis in a single incidence of treatment.

In the event that it is wished to utilise a fragment or derivative of sFRP3 a therapeutically effective amount may be between approximately 2.6 fmol and 40 pmol of the fragment or derivative per centimetre of wound or fibrosis (in a single incidence of treatment). Preferably a therapeutically effective amount of a fragment or derivative of sFRP3 may be between about 0.026 pmol and 26 pmol per centimetre of wound or fibrosis; more preferably between about 0.26 pmol and 26 pmol; more preferably still between about 0.26 pol and 2.6 pmol and most preferably about 0.26 pmol of the fragment or derivative per centimetre of wound or fibrosis in a single incidence of treatment.

It may be preferred that a therapeutically effective amount of sFRP3, or a fragment or derivative thereof, as considered in the preceding paragraphs be administered twice to a wound or site of fibrosis over a period of approximately 24 hours. The inventors believe that these therapeutically effective amounts (i.e. between approximately 5.2fmol and 80 pmol; preferably between about 0.052 pmol and 52 pmol; more preferably between about 0.52 pmol and 52 pmol; more preferably still between about 0.52 pol and 5.2 pmol and most preferably about 0.52 pmol) may also constitute preferred therapeutically effective amounts to be administered over the course of an entire regime of treatment.

Preferred therapeutically effective amounts of sFRP3, or a therapeutically effective fragment or derivative thereof, (either generally, or with reference to specific selected fragments or derivatives) may be investigated using in vitro and in vivo models, and suitable assessments of efficacy made with reference to various parameters for the measurement of scarring, as described elsewhere in the specification.

In the event that a fragment or derivative of sFRP3 comprises a different number of receptor binding sites to the number of receptor binding sites found in native sFRP3, this may alter the number of moles of such a fragment or derivative required in order to provide a therapeutically effective amount. For example, in the event that a derivative of sFRP3 comprises twice the number of binding sites present in native sFRP3, the amount of the derivative that will be needed to provide a therapeutically effective amount will generally be half of the amount(s) suggested above. Other such variations will be readily apparent to the skilled person.

The skilled person will appreciate that the suggestions above are provided for guidance. In particular it will be appreciated that the amount of sFRP3, or a therapeutically effective fragment or derivative thereof, to be administered via topical administration may be altered depending on permeability of the tissue or organ to which the topical composition is administered. Thus, in the case of relatively impermeable tissues or organs, it may be preferred to increase the amount of sFRP3, or a therapeutically effective fragment or derivative thereof, to be administered. Such an increased amount of sFRP3, or fragment or derivative thereof, may still represent a therapeutically effective amount, if the amount of the agent taken up into the tissue or organ where scarring is to be inhibited is therapeutically effective (i.e. if a therapeutically effective amount permeates the tissue or organ where scarring is to be inhibited, irrespective of the fact that a larger, non-therapeutic, amount of the agent may remain on the surface of, and unable to penetrate, the tissue or organ being treated).

It will be appreciated that the guidance provided herein, as to doses and amounts of an active agent to be used, is applicable both to medicaments of the invention, and also to the methods of the invention.

The inventors have found that sFRP3 may particularly preferably be administered in the form of a 10 ng/100 μl injectable solution, with 100 μl of such a solution provided per centimetre of wound or fibrosis in a 24 hour period. The solution may preferably be provided by intradermal injection.

In the case where the paragraphs above consider the administration of a specified amount of a medicament per linear cm of a wound it will be appreciated that this volume may be administered to either one or both of the margins of a wound to be treated (i.e. in the case of a reference to 100 μl of a medicament, this may be administered as 100 μl to the wound margins, or as 50 μl to each of the wound margins to be joined together).

The skilled person will recognise that the information provided in the preceding paragraphs as to amounts of sFRP3, or a therapeutically effective fragment or derivative thereof, which may be administered to wounds or sites of fibrotic disorders in order to inhibit scarring, may be varied by the skilled practitioner in response to the specific clinical requirements of an individual patient. These requirements may be determined by a range of factors including (but not limited to) the nature of the tissue to be treated, the area and/or depth of the wound or fibrosis to be treated, the severity of the wound or fibrosis, and the presence or absence of factors predisposing to pathological scar formation. For example, it will be appreciated that in the case of particularly deep or wide wounds the amounts provided by way of guidance above may be varied upwards, while still providing a therapeutically effective amount of sFRP3, or a fragment or derivative thereof. Suitable variations based on the guidance provided above will be readily apparent to those of skill in the art.

Centimetre of Wound or Fibrosis

In the context of the present disclosure, a “centimetre of wound” or a “centimetre of fibrosis” represents a unit by which the size of site at which scarring is to be prevented, reduced or inhibited may be measured. For the present purposes, a centimetre of wound may be taken to encompass a site where a wound is to be formed, as well as a wounded site, or both margins of a wounded site (should such margins exist).

A centimetre of wound in the context of the present disclosure constitutes a unit by which the size of a wound to be treated may be measured. A centimetre of wound may be taken to comprise any square centimetre of a body surface that is wounded in whole or in part. For example, a wound of two centimetres length and one centimetre width (i.e. with a total surface area of two centimetres²) will be considered to constitute “two wound centimetres”, while a wound having a length of two centimetres and a width of two centimetres (i.e. a total surface area of four centimetres²) will constitute four wound centimetres. By the same token, a linear wound of two centimetres length, but of negligible width (i.e. with negligible surface area), will, for the purposes of the present invention, be considered to constitute “two wound centimetres”, if it passes through two square centimetres of the body surface.

A centimetre of fibrosis should be construed in a similar manner, i.e. to encompass any square centimetre of the body in which scarring has occurred (either as a result of a fibrotic disorder, or the healing of a wound), as well as any square centimetre in which scarring may be expected to occur as a result of a fibrotic disorder.

The size of a site in wound centimetres, or centimetres of fibrosis, should generally be assessed when the wound is in its relaxed state (i.e. when the body site bearing the site to be measured is in the position adopted when the body is at rest). In the case of the skin, the relevant size should be assessed when the skin is not subject to external tension.

“Active Agents”

An “active agent”, for the purposes of the present disclosure, should be taken to be sFRP3, or any therapeutically effective fragment or derivative thereof. An active agent should also be taken to encompass any agent that promotes the expression or activity of sFRP3 or a therapeutically effective fragment or derivative thereof.

The skilled person will appreciate that a mixture of two, or more, different active agents may be used in the medicaments or methods of the invention to inhibit scarring. Indeed, such use may represent a preferred embodiment of the invention.

The skilled person will appreciate that many of the active agents suitable for use in the medicaments or methods of the present invention are suitable for cellular expression at a site where scarring is to be inhibited (or at a site from where their product may be available to a site where scarring is to be inhibited). This method of action may be termed “gene therapy”, and is described in greater detail elsewhere in the specification. In light of the above it will be appreciated that the cellular expression of a therapeutically effective amount of sFRP3, or a fragment or derivative thereof, at a site where scarring is to be inhibited represents a preferred embodiment of the invention. Such expression may preferably be transient, and may finish once a desired inhibition of scarring has been effected. Nucleic acid constructs encoding sFRP3, or a therapeutically effective fragment or derivative thereof, may be used in the medicaments or methods of the invention. Cells comprising nucleic acid constructs encoding sFRP3, or its therapeutically effective fragments or derivatives, may also be used in the medicaments or methods of the invention.

“Medicaments of the Invention”

For the purposes of the present disclosure, medicaments of the invention should be taken as encompassing any medicament manufactured in accordance with any aspect or embodiment of the invention.

Medicaments of the invention will generally comprise a pharmaceutically acceptable excipient, diluent or carrier in addition to the sFRP3, or therapeutically effective fragment or derivative thereof. Medicaments of the invention may, alternatively or additionally, comprise nucleic acid constructs encoding sFRP3 (or a therapeutically effective fragment or derivative thereof), or cells comprising such constructs.

Medicaments of the invention may preferably be in the form of an injectable solution comprising sFRP3, or a therapeutically effective fragment or derivative thereof. Solutions suitable for localised injection (and in particular for intradermal injection) constitute particularly preferred forms of the medicaments of the invention.

Preferred Body Sites

The inventors believe that the prevention, reduction or inhibition of scarring using sFRP3, or therapeutically effective fragments or derivatives thereof, can be effected at any body site and in any tissue or organ. The skin represents a preferred site at which scarring may be prevented, reduced or inhibited utilising the medicaments or methods of the invention. Without wishing to limit the scope of the invention, the following passages provide guidance as to specific tissues and body sites that may benefit from inhibition of scarring using the medicaments or methods of the invention.

The use of methods and medicaments of the invention to inhibit scarring may bring about a notable improvement in the cosmetic appearance of an injured area thus treated. Cosmetic considerations are important in a number of clinical contexts, particularly when scars may be formed at prominent body sites such as the face, neck and hands. Consequently it is a further preferred embodiment that the medicaments and methods of the invention be used to inhibit scarring at sites where it is desired to improve the cosmetic appearance of a scar formed.

In addition to its cosmetic impact, scarring of the skin is responsible for a number of deleterious effects afflicting those suffering from such scarring. For example, scarring of the skin may be associated with reduction of physical and mechanical function, particularly in the case of contractile scars (such as hypertrophic scars) and/or situations in which scars are formed across joints. The contraction exhibited by contractile scars of this kind is more pronounced than wound contraction that occurs as a normal part of the healing process, and may be distinguished from such normally occurring contraction in that it continues long after the healing process has ended (i.e. after wound closure). In cases of scars located in the area of joints the altered mechanical properties of scarred skin, as opposed to unscarred skin, and the effects of scar contraction may lead to dramatically restricted movement of a joint (articulation) so effected. Accordingly, it is a preferred embodiment that suitable medicaments and methods of the invention be used to inhibit scarring covering joints of the body (whether such scars result from the healing of wounds covering the joint, or are associated with fibrotic disorders covering the joint). In another preferred embodiment suitable medicaments and methods of the invention may be used to inhibit scarring at increased risk of forming a contractile scar (in the case of scarring that results from the healing of wounds this may include wounds of children, and/or wounds produced by burns).

The extent of scar formation, and hence extent of cosmetic or other impairment that may be caused by the scar, may also be influenced by factors such as the tension of the site at which the scar is formed (and in the case of scarring that results from the healing of a wound, the tension at the site where the wound is formed). For example, it is known that skin under relatively high tension (such as that extending over the chest, or associated with lines of tension) may be prone to formation of more severe scars than at other body sites. Thus in a preferred embodiment suitable medicaments and methods of the invention may be used to inhibit scarring at sites of high skin tension. The medicaments and methods of the invention may, for example, be used to inhibit scarring that results from healing of wounds located at sites of high skin tension.

It will be appreciated that tissues other than the skin may also be subject to scarring, whether associated with wounds or fibrotic disorders. The medicaments and methods of the invention may also be of benefit in inhibiting scarring associated with wounds or fibrotic disorders in these tissues.

The healing of wounds involving the peritoneum (the epithelial covering of the internal organs, and/or the interior of the body cavity) may frequently give rise to adhesions. Such adhesions are formed by bands of fibrous scar tissue, and can connect the loops of the intestines to each other, or the intestines to other abdominal organs, or the intestines to the abdominal wall. Adhesions can pull sections of the intestines out of place and may block passage of food. Adhesions are also a common sequitur of surgery involving gynaecological tissues. Incidences of adhesion formation may be increased in wounds that are subject to infection (such as bacterial infection) or exposure to radiation.

The medicaments or methods of the invention are suitable for use in the inhibition of scarring in the eye, and their use in this context represents a preferred embodiment of the invention. The inventors believe that the medicaments or methods of the invention may be used to inhibit scarring that results from healing of wounds to the eye, and/or to inhibit scarring associated with fibrotic disorders of the eye. Merely by way of example, the medicaments or methods of the invention may be used to inhibit scarring associated with glaucoma filtration surgery, corneal surgery (such as photorefractive keratectomy PRK, laser epithelial keratomileusis—LASEK, or laser assisted in situ keratomileusis—LASIK), cataract surgery (where scarring may frequently be associated with contraction of the lens capsule), or conjunctival cicatrisation.

Scarring in the central and peripheral nervous system may be inhibited using the medicaments of the invention. Such scarring may arise as a result of surgery or trauma and may additionally be assessed by future assays of nerve function e.g. sensory or motor tests. Inhibitors of scarring should improve such future outcomes.

Scarring in the blood vessels e.g. following anastomotic surgery, can lead to myointimal hyperplasia and reduction in the volume of the blood vessel lumen (restenosis). A therapeutically effective amount of sFRP3, or a therapeutically fragment or derivative thereof, may be provided to blood vessels by any suitable means.

The medicaments or methods of the invention may be used to inhibit scarring in tendons and ligaments. Such scarring may otherwise be expected to occur following surgery or trauma involving tissues of this type.

Agents of the invention may be used to inhibit scarring in a range of “internal” wounds or fibrotic disorders (i.e. wounds or fibrotic disorders occurring within the body, rather than on an external surface). Examples of internal wounds include penetrative wounds that pass through the skin into underlying tissues, and wounds associated with surgical procedures conducted within the body.

Preferred Wounds

The inventors believe that the medicaments or methods of the invention may be used to beneficially inhibit scarring in all types of wounds.

Examples of specific wounds in which scarring may be inhibited using the medicaments and methods of the invention include, but are not limited to, those independently selected from the group consisting of: wounds of the skin; wounds of the eye (including the inhibition of scarring resulting from eye surgery such as LASIK surgery, LASEK surgery, PRK surgery, or cataract surgery—in which the lens capsule may be subject to scarring), such as those giving rise to corneal cicatrisation; wounds subject to capsular contraction (which is common surrounding breast implants); wounds of blood vessels; wounds of the central and peripheral nervous system (where prevention, reduction or inhibition of scarring may enhance neuronal reconnection); wounds of tendons, ligaments or muscle; wounds of the oral cavity, including the lips and palate (for example, to inhibit scarring resulting from treatment of cleft lip or palate); wounds of the internal organs such as the liver, heart, brain, digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision). It is particularly preferred that the medicaments and methods of the invention be used to prevent, reduce or inhibit scarring associated with wounds of the skin. It may be preferred that the medicaments or methods of the invention be used to prevent scarring in tissues other than the heart.

The inventors believe that the ability of the medicaments and methods of the invention to inhibit scarring may reduce the occurrence of adhesions (such as those occurring in the abdomen, pelvis, thorax or spine). Accordingly, the use of medicaments or methods of the invention to prevent the formation of adhesions represents a preferred embodiment of the invention. The use of medicaments or methods of the invention in the inhibition of scarring involving the peritoneum is another preferred embodiment.

The medicaments and methods of the invention may be useful in the inhibition of scarring that may occur on healing of infected wounds or wounds exposed to radiation.

Incisional wounds constitute preferred wounds scarring resulting from which may be inhibited by the medicaments and methods of the invention. Surgical incisional wounds may constitute a particularly preferred group of wounds in respect of which scarring may be inhibited utilising the medicaments and methods of the invention..

It is a preferred embodiment that the medicaments and methods of the invention be used to inhibit scarring associated with cosmetic surgery. Since the great majority of cosmetic surgeries consist of elective surgical procedures it is readily possible to administer a therapeutically effective amount of sFRP3, or a fragment or derivative thereof, prior to surgery, and/or immediately following closure of the wound (e.g. with sutures), and this use represents a particularly preferred embodiment of the invention. In the case of elective surgical procedures a preferred route by which sFRP3, or a therapeutically effective fragment or derivative thereof, may be administered is via intradermal injection. Such injections may form raised blebs, which may then be incised as part of the surgical procedure, or alternatively the bleb may be raised by injecting the wound margins after the wound has been closed e.g. by sutures.

The cosmetic outcome of surgical procedures is also an important consideration in plastic surgery, and the use of methods or medicaments of the invention to inhibit scarring associated with plastic surgery constitutes a further preferred embodiment of the invention.

There are many surgical procedures that may be used in scar revision to allow realignment of wounds and scars such that they are subject to reduced tension. Probably the best known of these is “Z-plasty” in which two V-shaped flaps of skin are transposed to allow rotation of a line of tension. In a more preferred embodiment the medicaments and methods of the invention may be used to inhibit scarring of wounds during surgical revision of disfiguring scars.

Although individuals already subject to pathological scarring may suffer from a predisposition to further excessive scar formation, it is often clinically necessary to surgically revise hypertrophic scars or keloids, with an attendant risk of consequential pathological scar formation. Thus, it is a further preferred embodiment of the invention that the medicaments or methods herein described be used to inhibit scarring that results from wounds produced by surgical revision of pathological scars.

Pathological scarring may have more pronounced deleterious effects than arise even as a result of relatively severe normal scarring. Common examples of pathological scars include keloids, hypertrophic scars and pterygium. It is recognised that certain types of wound, or certain individuals may be predisposed to pathological scar formation. For instance individuals of the African Continental Ancestry Group or Asian Continental Ancestry Group, or those having a familial history of pathological scarring may be considered to be at increased risk of hypertrophic scar or keloid formation. Wounds of children, and particularly burns wounds of children, are also associated with increased hypertrophic scar formation. Incidences of pterygium may be increased amongst those in whom the eye is frequently exposed to intense sunlight or dust. Accordingly it is a preferred embodiment of the invention that suitable medicaments and methods be used to inhibit scarring of wounds in which there is an increased risk of pathological scar formation.

Keloid scars (or keloids) constitute a notable example of pathological scarring, and are raised scars that spread beyond the margins of the original wound and invade the surrounding normal skin. Keloids continue to grow over time, do not regress spontaneously, and frequently recur following surgical excision. Keloid scars occur with equal frequency in men and women, mainly from ages 10 to 30, and can result from piercing, surgery, vaccination, tattoos, bites, blunt trauma and burns. A number of studies have suggested that there is an underlying genetic predisposition to keloid formation since keloid scars are more prevalent in dark skinned races, and in individuals of the African Continental Ancestry Group or Asian Continental Ancestry Group.

Keloids appear as elevated scars that may typically be hyperpigmented or hypopigmented in relation to the surrounding skin. Keloids may be characterised on the basis of their tendency to grow beyond the initial boundaries of the wound from which they result. At a microscopic level, keloids may be characterised by the presence of large whorls of collagen, and the predominantly acellular nature of the interior of the lesion.

Hypertrophic scars are raised scars which may have an appearance very similar to keloid lesions. Unlike keloids, hypertrophic scars do not expand beyond the boundaries of the original injury and are not prone to recurrence after excision. Hypertrophic scars may frequently undergo contraction, and it is believed that the contractile nature of hypertrophic scars may be associated with the elevated numbers of myofibroblasts that are frequently reported within these types of scars. Hypertrophic scars may commonly arise as a result of burn or scald injuries, and are particularly common amongst children.

Pterygium is a hypertrophied outgrowth of the subconjunctival tissue to the border of the cornea or beyond. The outgrowth is typically triangular in shape, with the apex pointing towards the pupil. Pterygium may interfere with vision, and may require surgery to remove the hypertrophied tissue. Furthermore, the tissue may frequently re-grow after excision, in the same manner as keloid scars, thus requiring multiple incidences of surgery.

It is recognised that wounds resulting from burns injuries (which for the purposes of the present invention may be taken to encompass scalding injuries involving hot liquids or gasses; “freezer burn” injuries caused by exposure to extreme low temperatures; radiation burns; and chemical burns, such as those caused by caustic agents) may extend over great areas of an individual so afflicted. Accordingly, burns may give rise to scar formation covering a large proportion of a patient's body. This great extent of coverage increases the risk that the scar formed will cover areas of elevated cosmetic importance (such as the face, neck, arms or hands) or of mechanical importance (particularly the regions covering or surrounding joints). Burns injuries caused by hot liquids are frequently suffered by children (for example as a result of upsetting pans, kettles or the like) and, due to the relatively smaller body size of children, are particularly likely to cause extensive damage over a high proportion of the body area. Furthermore, burns injuries, and particularly those suffered by children, have an elevated risk of producing pathological hypertrophic scars of the type described below. Such hypertrophic scars may increase both the cosmetic and mechanical impairment associated with scarring after burns. Accordingly, it is a preferred embodiment that medicaments and methods of the invention be used to inhibit scarring resulting from burns injuries.

The ability of sFRP3, or therapeutically effective fragments or derivatives thereof, to inhibit scarring is of great utility in the inhibition of scarring associated with grafting procedures. In particular, the medicaments and methods of the invention may be used to inhibit scarring that results from wounds associated with grafting procedures. Inhibition of scarring using the medicaments and methods of the invention is of benefit both at a graft donor sites and graft recipient sites. The scar inhibitory effects of the medicaments and methods of the invention are able to inhibit scarring that may otherwise occur at sites where tissue for grafting is removed, or that may be associated with the healing and integration of grafted tissue. The inventors believe that the methods and medicaments of the invention confer advantages in the inhibition of scarring that may otherwise be associated with grafts utilising skin, artificial skin, or skin substitutes.

The inventors also believe that the medicaments and methods of the invention may be used to inhibit scarring associated with encapsulation. Encapsulation is a form of scarring that occurs around sites at which implant materials (such as biomaterials) have been introduced into the body. Encapsulation is a frequent complication associated with breast implants, and the use of the medicaments or methods of the invention to inhibit encapsulation in this context is a preferred embodiment of the invention.

The medicaments or methods of the invention may be of benefit in inhibiting scarring in the eye (and particularly in the cornea or retina). Scarring of the cornea may result from corneal wounds, which may be caused by trauma to the cornea arising as a result of accidental injury or as a result of surgical operations such as LASIK, LASEK or PRK procedures. Scarring elsewhere in the eye, such as at sites of pressure relieving blebs formed in glaucoma surgery, or scarring of the retina associated with proliferative vitreoretinopathy may also be inhibited by the medicaments and methods of the present invention.

The medicaments and methods of the invention may be used to inhibit scarring that results from healing of wounds selected from the group consisting of: (also commonly referred to as “scrapes”, these are shallow injuries which frequently cover a relatively large area); avulsions (when an entire bodily structure, or a part of such a structure, is forcibly pulled away from its site); crush wounds; incisional wounds; lacerations; punctures; and missile wounds. All of these different types of wounds may be suffered by the skin, among other tissues or organs, and all may, to a greater or lesser extent, result in scarring.

The wounds resulting from surgical procedures are most commonly incisional wounds, and these are a frequent cause of scarring. Accordingly it is a preferred embodiment that the medicaments and methods of the invention be used in the inhibition of scarring resulting from incisional wounds, such as surgical wounds.

The inventors believe that the medicaments or methods of the invention may be of use in inhibiting scarring associated with full thickness or partial thickness wounds (respectively wounds in which the epithelial layer is either totally or partly compromised). Preferred examples of partial thickness wounds, scarring associated with which may be inhibited using the medicaments or methods of the invention, include “skin peels” such as “chemical peels” (such as alphahydroxy acid peels, trichloroacetic acid peels or phenol peels) or “laser peels”; wounds associated with dermabrasion; and wounds associated with dermaplaning. It may particularly be preferred that the medicaments or methods of the invention be used to inhibit scarring associated with partial thickness wounds occurring at cosmetically important sites (such as the face), which may frequently be the subject of skin peel treatment.

Preferred Fibrotic Disorders

The medicaments or methods of the invention may be used to prevent, reduce or inhibit scarring associated with any fibrotic disorder. By way of example, and without limiting the scope of protection sought, the medicaments or methods of the invention may preferably be used to treat fibrotic disorders independently selected from the group consisting of skin fibrosis; scleroderma; progressive systemic fibrosis; lung fibrosis; muscle fibrosis; kidney fibrosis; glomerulosclerosis; glomerulonephritis; uterine fibrosis; renal fibrosis; cirrhosis of the liver, liver fibrosis; chronic obstructive pulmonary disease; fibrosis following myocardial infarction; central nervous system fibrosis, such as fibrosis following stroke; fibrosis associated with neuro-degenerative disorders such multiple sclerosis; fibrosis associated with proliferative vitreoretinopathy (PVR); restenosis; endometriosis; ischemic disease and radiation fibrosis.

Prevention, Reduction or Inhibition of Scarring

The prevention, reduction or inhibition of scarring within the context of the present invention should be understood to encompass any degree of prevention, reduction or inhibition in scarring achieved on healing of a treated wound, or in a treated scar or treated site of a fibrotic disorder as compared to the level of scarring occurring on healing of a control-treated or untreated wound, or in an untreated scar, or at an untreated site of a fibrotic disorder. Throughout the specification references to “prevention”, “reduction” or “inhibition” of scarring are generally to be taken, except where the context requires otherwise, to represent substantially equivalent activities, involving equivalent mechanisms mediated by sFRP3, or its therapeutically effective fragments or derivatives, and that are all manifested in anti-scarring activity.

For the sake of brevity, the present specification will primarily refer to “inhibition” of scarring utilising sFRP3, or therapeutically effective fragments or derivatives thereof. However, such references should be taken, except where the context requires otherwise, to also encompass the prevention or reduction of scarring utilising such active agents. Similarly, references to “prevention” of scarring using sFRP3, or its therapeutically effective fragments or derivatives should, except where the context requires otherwise, be taken also to encompass the treatment of scarring using such active agents.

The inhibition of scarring achieved using methods and medicaments of the invention may be assessed and/or measured with reference to the microscopic and/or macroscopic appearance of a treated scar. Inhibition of scarring may also suitably be assessed with reference to microscopic and/or macroscopic appearance of a treated scar as compared to the microscopic and/or macroscopic appearance of an untreated scar.

Suitable methods and parameters by which the scarring (and hence any inhibition of scarring) may be assessed in treated scars or control scars are described elsewhere in the specification, as are methods by which such assessments may be captured and quantified (if so required).

“Treated Wounds”, “Untreated Wounds”, “Treated sites of Fibrosis”, “Untreated Sites of Fibrosis”, “Treated Scars” and “Untreated Scars”

Treatment of wounds with a therapeutically effective amount of sFRP3, or of a fragment or derivative thereof, is able to inhibit the scarring that may otherwise be expected to occur on healing of untreated wounds.

For present purposes an “untreated wound” should be considered to be any wound that has not been exposed to a therapeutically effective amount of sFRP3, or a therapeutically effective fragment or derivative thereof. A “diluent control-treated wound” will be an untreated wound to which a control diluent has been administered, and a “naïve control” will be an untreated wound made without administration of sFRP3, or a therapeutically effective fragment or derivative thereof, and without a suitable control diluent, and left to heal without therapeutic intervention.

In contrast, a “treated wound” may be considered to be a wound exposed to a therapeutically effective amount of sFRP3, or a fragment or derivative thereof. Thus a treated wound may be a wound which has been provided with a medicament of the invention, or which has received treatment in accordance with the methods of the invention.

For the present purposes a “treated scar” should be taken to encompass:
i) a scar that results from healing of a treated wound (i.e. a wound treated with a therapeutically effective amount of sFRP3, or a fragment or derivative thereof); and/or
ii) a scar produced at a site of a fibrotic disorder that has been treated with a therapeutically effective amount of sFRP3, or a fragment or derivative thereof; and/or
iii) a scar to which a therapeutically effective amount of sFRP3, or a fragment or derivative thereof, has been administered.

By way of contrast, an “untreated scar” should be taken to encompass:
i) a scar that results from healing of an untreated wound (for example a wound treated with a placebo, control, or standard care); and/or
ii) a scar to which a therapeutically effective amount of sFRP3, or a therapeutically effective fragment or derivative thereof, has not been administered.

Untreated scars may typically be used as comparators in assessing the inhibition of scarring that may be evident in a treated scar. Suitable comparator untreated scars of this type may preferably be matched to the treated scar with reference to one or more criteria independently selected from the group consisting of: scar age; scar size; scar site; patient age; patient race and patient gender.

Treatment of a site of a fibrotic disorder with a therapeutically effective amount of sFRP3, or of a fragment or derivative thereof, is able to inhibit scarring, and will give rise to a “treated site of fibrosis”, which will comprise a treated scar. The scarring at such a treated site of fibrosis may be compared with that occurring in an untreated or control site of a fibrotic disorder (i.e. a site which has not been provided with a therapeutically effective amount of sFRP3, or a fragment or derivative thereof).

The inventors believe that treatment of fibrotic disorders in this manner may have an impact on the macroscopic and/or microscopic appearance of the scar associated with the fibrotic disorder, such that the macroscopic and/or microscopic structure of a scar at a treated site of fibrosis will be more akin to that found in normal non-fibrotic tissue. For example, in the case of fibrosis involving the skin, a treated scar may, when viewed microscopically, exhibit an abundance and orientation of ECM molecules, such as collagen, that is more similar to that found in normal skin than that found in untreated scars.

Models of Scarring

In the case of inhibition of scarring that results from the healing of a wound, a suitable animal model in which the therapeutic effectiveness of sFRP3, or a fragment or derivative thereof, may be assessed, and in which a therapeutically effective amount of an active agent may be determined, may involve providing the sFRP3, or fragment or derivative thereof, to incisional or excisional wounds of experimental subjects (either humans subjects, or non-human animals such as mice, rats or pigs), and assessing the scarring that results on healing of the wound.

In the case of inhibition of scarring associated with fibrotic disorders, the commonality of the biological mechanisms underlying scarring means that this scarring may also be investigated using incisional or excisional wound healing models of the type outlined above.

However, the skilled person will also be aware of specific experimental models of fibrotic disorders that may be used to further investigate the therapeutic effectiveness of sFRP3, or therapeutically effective fragments or derivatives thereof, in this context. For example, administration of bleomycin to lungs or skin of experimental animals allows the generation of an experimental model of fibrosis of the lung or skin that may be used to assess effectiveness of sFRP3, or a fragment or derivative thereof, in the context of inhibiting scarring associated with lung fibrosis or dermal fibrosis. The administration of CCl₄ to experimental animals allows the generation of an experimental model of fibrosis of the liver that may be used to assess effectiveness of sFRP3, or a fragment or derivative thereof, in the context of inhibiting scarring associated with liver fibrosis. Furthermore, an experimental model of glomerulonephritis may be established either by injection of suitable serum proteins into an experimental animal or injection of nephrotoxic serum, and either of these animal models may be useful in assessment of sFRP3, or fragments or derivatives thereof, in the inhibition of scarring associated with kidney fibrosis.

The experimental models described above may also allow identification of particular effective routes or regimes by which sFRP3, or its therapeutically effective fragments or derivatives, may be administered. These routes or regimes may provide notable advantages in the context of the medicaments and methods of the present invention, and these may give rise to further aspects of the invention.

Assessment of Scarring, and of Inhibition of Scarring

The extent of inhibition of scarring that may be required in order to achieve a therapeutic effect will be apparent to, and may readily be determined by, a clinician responsible for the care of the patient. The clinician may undertake a suitable determination of the extent of inhibition of scarring that has been achieved using sFRP3, or a therapeutically effective fragment or derivative thereof, in order to assess whether or not a therapeutic effect has been achieved, or is being achieved. Such an assessment may, but need not necessarily, be made with reference to suggested methods of measurement described herein.

The extent to which inhibition of scarring utilising sFRP3, or a therapeutically effective fragment or derivative thereof is achieved may be assessed with reference to the effects that such an active agent may achieve in human patients treated with the methods or medicaments of the invention. Alternatively, inhibition of scarring that may be achieved by sFRP3, or a therapeutically effective fragment or derivative thereof, may be assessed with reference to experimental investigations using suitable in vitro or in vivo models. The use of experimental models to investigate inhibition of scarring may be particularly preferred in assessing the therapeutic effectiveness of particular fragments or derivatives of sFRP3, or in establishing therapeutically effective amounts of such fragments or derivatives.

Animal models of scarring represent preferred experimental models for in vivo assessment of the extent of scar inhibition that may be achieved using the medicaments or methods of the invention. Suitable models may be used specifically to investigate scarring that results from healing of a wound, and, additionally or alternatively, to investigate scarring associated with fibrotic disorders. Suitable models of both types will be known to those skilled in the art, and examples of such models are described elsewhere in the specification for illustrative purposes.

Inhibition of scarring, using the medicaments and methods of the invention, can be effected at any body site and in any tissue or organ so far investigated. For illustrative purposes the scar inhibitory activity of medicaments and methods of the invention will primarily be described with reference to inhibition of scarring that may be brought about in the skin (the body's largest organ). However, the skilled person will immediately appreciate that many of the factors that are relevant when considering inhibition of scarring in the skin are also relevant to inhibition of scarring in other organs or tissues. Accordingly the skilled person will recognise that, except for where the context requires otherwise, the parameters and assessments considered below in respect of scars of the skin may also be applicable to scarring in tissues other than the skin.

In the skin, treatment may improve the macroscopic and microscopic appearance of scars; macroscopically the scars may be less visible and blend with the surrounding skin, microscopically the collagen fibres within the scar may have morphology and organisation that is more similar to those in the surrounding skin.

The inhibition of scarring achieved using methods and medicaments of the invention may be assessed and/or measured with reference to either the microscopic or macroscopic appearance of a treated scar as compared to the appearance of an untreated scar. Inhibition of scarring may also suitably be assessed with reference to both macroscopic and microscopic appearance of a treated scar.

In considering the macroscopic appearance of a scar resulting from a treated wound, the extent of scarring, and hence the magnitude of any inhibition of scarring achieved, may be assessed with reference to any of a number of parameters. Most preferably, holistic assessment of the scar by means of assessment of macroscopic photographs by an independent expert panel, by means of an independent lay panel or clinically by means of a macroscopic assessment by a clinician of the patients themselves. Assessments are captured by means of a VAS (visual analogue scale) or a categorical scale.

Macroscopic characteristics of a scar which can be assessed objectively include:
i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Similarly, scars may be redder than the surrounding skin. In this case inhibition of scarring may be demonstrated when the redness of a treated scar fades earlier, or more completely, or to resemble more closely the appearance of the surrounding skin, compared to an untreated scar. There are a number of non-invasive colorimetric devices which are able to provide data with respect to pigmentation of scars and unscarred skin, as well as redness of the skin (which may be an indicator of the degree of vascularity present in the scar or skin). Examples of such devices include the X-rite SP-62 spectrophotometer, Minolta Chronometer CR-200/300; Labscan 600; Dr. Lange Micro Colour; Derma Spectrometer; laser-Doppler flow meter; and Spectrophotometric intracutaneous Analysis (SIA) scope.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on a patient by means of profilometry, or indirectly, by profilometry of moulds taken from a scar.
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can be measured directly on a patient by means of profilometry, or indirectly by profilometry of moulds taken from a scar.
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two parameters, even more preferably at least three parameters, and most preferably at least four of these parameters (for example, all four of the parameters set out above). The parameters described above may be used in the development of a visual analogue scale (VAS) for the macroscopic assessment of scarring. Details regarding implementation of VASs are described below.

Microscopic assessment may also provide a suitable means by which the quality of treated and untreated or control scars may be compared. Microscopic assessment of scar quality may typically be carried out using histological sections of scars. Suitable parameters for the microscopic assessment of scars may include:
i) Thickness of extracellular matrix (ECM) fibres. Scars typically contain thinner ECM fibres than are found in the surrounding skin. This property is even more pronounced in the case of keloid and hypertrophic scars. Inhibition of scarring may be demonstrated when the thickness of ECM fibres in a treated scar more closely approximates the thickness of ECM fibres found in unscarred skin than does the thickness of fibres found in an untreated scar.
ii) Orientation of ECM fibres. ECM fibres found in scars tend to exhibit a greater degree of alignment with one another than do those found in unscarred skin (which have a random orientation frequently referred to as “basket weave”). The ECM of pathological scars such as keloids and hypertrophic scars may exhibit even more anomalous orientations, frequently forming large “swirls” or “capsules” of ECM molecules. Accordingly, inhibition of scarring may be demonstrated when the orientation of ECM fibres in a treated scar more closely approximates the orientation of ECM fibres found in unscarred skin than does the orientation of such fibres found in an untreated scar.
iii) ECM composition of the scar. The composition of ECM molecules present in scars shows differences from that found in normal skin, with a reduction in the amount of elastin present in ECM of scars. Thus inhibition of scarring may be demonstrated when the composition of ECM fibres in the dermis of a treated scar more closely approximates the composition of such fibres found in unscarred skin than does the composition found in an untreated scar.
iv) Cellularity of the scar. Scars tend to contain relatively fewer cells than does unscarred skin. It will therefore be appreciated that inhibition of scarring may be demonstrated when the cellularity of a treated scar more closely approximates the cellularity of unscarred skin than does the cellularity of an untreated scar.

Other features that may be taken into account in assessing the microscopic quality of scars include elevation or depression of the scar relative to the surrounding unscarred skin, and the prominence or visibility of the scar at the interface with the unscarred skin

The parameters described above may be used in generating a VAS for the microscopic assessment of scarring. Such a VAS may consider collagen organisation and abundance in the papillary dermis and the reticular dermis may also provide a useful index of scar quality. Inhibition of scarring may be indicated when the quality of a treated scar is closer to that of unscarred skin than is the quality of an untreated or control scar.

It is surprising to note that the overall appearance of scars, such as those of the skin, is little influenced by the epidermal covering of the scar, even though this is the part of the scar that is seen by the observer. Instead, the inventors find that the properties of the connective tissue (such as that making up the dermis, or neo-dermis) present within the scar have greater impact on the perception of extent of scarring, as well as on the function of the scarred tissue. Accordingly assessments of criteria associated with the connective tissues such as the dermis, rather than epidermis, may prove to be the most useful in determining inhibition of scarring.

The thickness of ECM fibres and orientation of ECM fibres may be favoured parameters, for assessing inhibition of scarring. A treated scar may preferably have improved ECM orientation (i.e. orientation that is more similar to unscarred skin than is the orientation in an untreated scar).

A treated scar will preferably demonstrate inhibition of scarring as assessed with reference to at least one of the parameters for microscopic assessment set out above. More preferably a treated scar may demonstrate inhibition of scarring with reference to at least two of the parameters, even more preferably at least three of the parameters, and most preferably all four of these parameters.

It will be appreciated that inhibition of scarring achieved using the medicaments or methods of the invention may be indicated by improvement of one or more suitable parameters combined from different assessment schemes (e g inhibition as assessed with reference to at least one parameter used in macroscopic assessment and at least one parameter used in microscopic assessment).

Further examples of suitable parameters for the clinical measurement and assessment of scars may be selected based upon a variety of measures or assessments including those described by Duncan et al. (2006), Beausang et al. (1998) and van Zuijlen et al. (2002). Except for where the context requires otherwise, many of the following parameters may be applied to macroscopic and/or microscopic assessment of scarring. Examples of Suitable parameters for assessment of scars in the skin may include:

1. Assessment with Regard to Visual Analogue Scale (VAS) Scar Score.

Prevention, reduction or inhibition of scarring may be demonstrated by a reduction in the VAS score of a treated scar when compared to a control scar. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar.

2. Assessment with Regard to a Categorical Scale.

Prevention, reduction or inhibition of scarring may be determined by allocating scars to different categories based on either textual descriptions e.g. “barely noticeable”, “blends well with normal skin”, “distinct from normal skin”, etc., by comparing a treated scar and a an untreated or control scar, noting any differences between these, and allocating the differences to selected categories (suitable examples of which may be “mild difference”, “moderate difference”, “major difference”, etc.). Assessment of this sort may be performed by the patient, by an investigator, by an independent panel, or by a clinician, and may be performed either directly on the patient or on photographs or moulds taken from the patient. Inhibition of scarring may be demonstrated when an assessment indicates that treated scars are generally allocated to more favourable categories than are untreated or control scars.

3. Scar Height, Scar Width, Scar Perimeter, Scar Area or Scar Volume.

The height and width of scars can be measured directly upon the subject, for example by use of manual measuring devices such as callipers, or automatically with the use of profilometers. Scar width, perimeter and area may be measured either directly on the subject, by image analysis of photographs of the scar, or using plaster casts of impressions of the scar. The skilled person will also be aware of further non-invasive methods and devices that can be used to investigate suitable parameters, including silicone moulding, ultrasound, optical three-dimensional profilimetry and high resolution Magnetic Resonance Imaging.

Inhibition of scarring may be demonstrated by a reduction in the height, width, area, perimeter or volume, or any combination thereof, of a treated scar as compared to an untreated scar.

4. Scar Distortion and Mechanical Performance

Scar distortion may be assessed by visual comparison of a scar and unscarred skin. A suitable comparison may categorise a selected scar as causing no distortion, mild distortion, moderate distortion or severe distortion.

The mechanical performance of scars can be assessed using a number of non-invasive methods and devices based upon suction, pressure, torsion, tension and acoustics. Suitable examples of devices capable of use in assessing mechanical performance of scars include Indentometer, Cutometer, Reviscometer, Visco-elastic skin analysis, Dermaflex, Durometer, Dermal Torque Meter and Elastometer.

Inhibition of scarring may be demonstrated by a reduction in distortion caused by treated scars as compared to that caused by untreated scars. It will also be appreciated that inhibition of scarring may be demonstrated by the mechanical performance, of unscarred skin being more similar to that of treated scars than of untreated scars.

Photographic Assessments

Independent Lay Panel

Photographic assessment of treated and untreated scars may be performed by an independent lay panel of assessors using standardised and calibrated photographs of the scars. The scars may be assessed by an independent lay panel to provide categorical ranking data (e.g. that a given treated scar is “better”, “worse” or “no different” when compared to an untreated scar) and quantitative data using a Visual Analogue Scale (VAS) based upon the method described by Duncan et al. (2006) and Beausang et al. (1998). The capture of these data may make use of suitable software and/or electronic system(s) as described in the applicant's co-pending patent application filed as PCT/GB2005/004787.

Expert Panel

Photographic assessment of treated and untreated scars may alternatively or additionally be performed by a panel of expert assessors using standardised and calibrated photographs of the scars to be assessed, and/or positive casts of silicone moulds. The panel of experts may preferably consist of individuals skilled in the art, suitable examples of which include plastic surgeons, dermatologists or scientists having relevant technical backgrounds.

Clinical Assessment

A clinician, or an independent panel of clinicians may assess the scar(s) on a patient using any of the forgoing parameters e.g. VAS, colour, categorical scales, etc. A suitable clinician may be a clinician responsible for care of a patient, or may be a clinician investigating efficacy of therapies for inhibition of scarring.

Patient Assessment

A patient may assess their own scars and/or compare scars by means of a structured questionnaire. A suitable questionnaire may measure parameters such as: the patient's satisfaction with their scar; how well the scar blends with the unscarred skin; as well as the effect of the scar on their daily life (suitable questions may consider whether the .patient uses clothes to hide the scar, or otherwise avoids exposing it) and/or scar symptoms (examples of which may include itch, pain or paresthesia). Inhibition of scarring may be indicated by the treated scar receiving a more positive rating from the patient, and/or causing the patient fewer problems, and/or causing fewer or less scar symptoms, and/or an increase in patient satisfaction compared to an untreated scar.

In addition to categorical data, quantitative data (preferably relating to the above parameters) can be generated using image analysis in combination with suitable visualisation techniques. Examples of suitable visualisation techniques that may be employed in assessing scar quality are specific histological stains or immuno-labelling, wherein the degree of staining or labelling present may be quantitatively determined by image analysis

Quantitative data may be usefully and readily produced in relation to the following parameters:

1. Scar width, height, elevation, volume and area.

2. Collagen organisation, collagen fibre thickness, collagen fibre density.

2. Number and orientation of fibroblasts.

4. Quantity and orientation of other ECM molecules e.g. elastin, fibronectin

Prevention, reduction or inhibition of scarring may be demonstrated by a change in any of the parameters considered above such that a treated scar more closely resembles unscarred skin than does a control or untreated scar (or other suitable comparator).

The assessments and parameters discussed above are suitable for assessment of the effects of sFRP3, or its fragments or derivatives, on scar formation, as compared to control, placebo or standard care treatment in animals or humans. It will be appreciated that these assessments and parameters may be utilised in determining therapeutically effective fragments or derivatives of sFRP3 that may be used for scar prevention, reduction or inhibition; and in determining therapeutically effective amounts of sFRP3, or its fragments or derivatives. Appropriate statistical tests may be used to analyse data sets generated from different treatments in order to investigate significance of results.

Many of the parameters described above for the assessment of scarring have previously been viewed as primarily suitable for the assessment of scarring that results from healing of a wound. However, the inventors believe that many of these parameters are also suitable for assessment of scarring associated with fibrotic disorders. Additional or alternative parameters that may be considered when assessing scarring associated with fibrotic disorders will be apparent to the skilled person. The following examples are provided by way of illustration only.

Scarring associated with fibrotic disorders may be assessed with reference to trichrome staining (for example Masson's trichrome or Mallory's trichrome) of biopsy samples taken from a tissue believed to be subject to the fibrotic disorder. These samples may be compared with non-scarred tissues that have been taken from tissues not subject to the fibrotic disorder, and with reference tissues representative of staining in the same tissue (or a range of tissues) subject to different extents of scarring associated with the fibrotic disorder. Comparisons of such tissues may allow assessment of the presence and extent of scarring associated with a fibrotic disorder that is present in the tissue of interest. Protocols for trichrome staining are well known to the skilled person, and kits that may be used to conduct trichrome staining are commercially available.

It will be appreciated that in many cases it may be preferred to avoid invasive procedures such as the collection of biopsies. In recognition of this fact a number of non-invasive procedures have been devised that allow assessment of scarring associated with fibrotic disorders without the need for biopsy samples. Examples of such procedures include Fibrotest (FT) and Actitest (AT).

These commercially available assays use five or six biochemical markers of scarring associated with fibrotic disorders for use as a non-invasive alternative to liver biopsy in patients with chronic hepatitis C or B, alcoholic liver disease and metabolic steatosis (for instance the overweight, patients with diabetes or hyperlipidemia). Through use of such biochemical markers, and analysis using selected algorithms, these procedures are able to determine levels of liver fibrosis and necroinflammatory activity. The use of such tests is increasingly clinically accepted as an alternative to biopsies, and the tests are commercially available from suppliers such as BioPredictive.

It will be appreciated by the skilled person that the methods described above may be used to allow assessment of scarring that is associated with one or more fibrotic disorders in order to determine whether or not prevention, reduction or inhibition of such scarring utilising the medicaments or methods of the invention would be advantageous. Furthermore, scar assessment methods of the type described above may be used to determine therapeutically effective fragments or derivatives of sFRP3 suitable for inhibition of scarring associated with a fibrotic disorder, as well as determining therapeutically effective amounts of sFRP3, or its fragments or derivatives.

Corneal scarring may be assessed by measuring the opacity, or transmitting/refractory properties, of the cornea. Such assessments may, for example, be made using in vivo confocal microscopy.

Successful inhibition of scarring in tendons or ligaments may be indicated by restoration of function of tissues treated with the medicaments or methods of the invention. Suitable indicia of function may include the ability of the tendon or ligament to bear weight, stretch, flex, etc.

The extent of scarring occurring in blood vessels can be measured directly e.g. using ultrasound, or indirectly by means of blood flow Inhibition of scarring achieved using the medicaments or methods of the invention may lead to a reduction in narrowing of the blood vessel lumen and allow a more normal blood flow.

Administration Regimes

The methods or medicaments of the invention may be used to provide a therapeutically effective amount of sFRP3, or a fragment or derivative thereof, to a site of existing scarring (whether as a result of a wound or fibrotic disorder), or to a site where scarring is likely to occur (for example a wound, or site of a fibrotic disorder, or a site where a wound or fibrotic disorder is likely to occur). Alternatively, the medicaments or methods of the invention may be used prophylactically, i.e. prior to scar formation. For example, methods or medicaments of the invention may be utilised prior to wounding or prior to the onset of a fibrotic disorder.

In the case of the inhibition of scarring associated with healing of a wound, prophylactic use may involve administration of a therapeutically effective amount of sFRP3, or fragments or derivatives thereof, at sites where no wound presently exists, but where a wound that would otherwise give rise to a scar is to be formed. By way of example, a therapeutically effective amount of sFRP3, or a fragment or derivative thereof, may be administered to sites that are to undergo wounding as a result of elective procedures (such as surgery), or to sites that are believed to be at elevated risk of wounding.

It may be preferred that the medicaments of the invention are administered to the site around the time of wounding, or immediately prior to the forming of a wound (for example in the period up to six hours before wounding) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may determined with reference to factors such as the patient's age, health, and predisposition to healing complications or adverse scarring). The prophylactic use of methods and medicaments in accordance with the invention is a preferred embodiment of the invention, and is particularly preferred in the prevention, reduction or inhibition of scarring in the context of surgical wounds.

In the case of the inhibition of scarring associated with fibrotic disorders, medicaments of the invention may be administered to a site at elevated risk of developing a fibrotic disorder prior to formation of said disorder. Suitable sites may be those that are perceived to be at elevated risk of the development of fibrotic disorders. An elevated risk of development of fibrotic disorders may arise as a result of disease, or as a result of environmental factors (including exposure to fibrotic agents), or as a result of genetic predisposition.

When used for the inhibition of scarring associated with fibrotic disorder, a therapeutically effective amount of sFRP3, or a fragment or derivative thereof, may be administered immediately prior to onset of a fibrotic disorder, or at an earlier time. The skilled person will be able to establish the optimal time for administration of medicaments of the invention used to treat fibrotic disorders using standard techniques well known to those skilled in the art, and familiarisation with the clinical progression of scarring associated with fibrotic disorders.

The methods and medicaments of the invention are also able to inhibit scarring if administered after a wound has already been formed. It is preferred that such administration should occur as early as possible after formation of the wound, but agents of the invention are able to inhibit scarring at any time up until the healing process has been completed (i.e. even in the event that a wound has already partially healed the methods and medicaments of the invention may be used to inhibit scarring in respect of the remaining un-healed portion). It will be appreciated that the “window” in which the methods and medicaments of the invention may be used to inhibit scarring is dependent on the nature of the wound in question (including the degree of damage that has occurred, and the size of the wounded area). Thus, in the case of a large wound, the methods and medicaments of the invention may be administered relatively late in the healing response yet still be able to inhibit scarring, as a consequence of the relatively prolonged time that large wounds require to heal.

The methods and medicaments of the invention may, for instance, preferably be administered within the first 24 hours after a wound is formed, but may still inhibit scarring if administered up to ten, or more, days after wounding.

Similarly, the methods and medicaments of the invention may be administered to a site at which a fibrotic disorder is already developing, in order to prevent further scarring associated with the fibrotic disorder taking place. This use will obviously be advantageous in situations in which the degree of scarring that has occurred prior to administration of sFRP3, or therapeutically effective fragment or derivative thereof, is sufficiently low that the fibrotic tissue is still able to function.

Medicaments of the invention may preferably be administered within 24 hours of the onset of scarring associated with a fibrotic disorder, but may still be effective if administered considerably later in the fibrotic process. For example, medicaments of the invention may be administered within a month of the onset of the fibrotic disorder (or of the diagnosis that scarring associated with the fibrotic disorder is taking place), or within sixth months, or even one or more years, depending on the extent of scarring that has already occurred, the proportion of the tissue effected by the fibrotic disorder, and the rate at which the fibrotic disorder is progressing.

The methods and medicaments of the invention may be administered on one or more occasions (as necessary) in order to inhibit scarring.

For instance, in the case of inhibition of scarring that results from the healing of a wound, therapeutically effective amounts of sFRP3, or a fragment or derivative thereof, may be administered to a wound as often as required until the healing process has been completed. By way of example, the medicaments of the invention may be administered daily or twice daily to a wound for at least the first three days following the formation of the wound. In a particularly preferred embodiment a medicament of the invention may be administered prior to wounding and again approximately 24 hours following wounding.

Most preferably the methods or medicaments of the invention may be administered both before and after formation of a wound. The inventors have found that administration of the medicaments of the invention immediately prior to the formation of a wound, followed by daily administration of sFRP3, or a therapeutically effective fragment or derivative thereof, for one or more days following wounding, is particularly effective in inhibiting scarring resulting from the healing of a wound, or associated with a fibrotic disorder.

In the case where sFRP3, or a therapeutically effective fragment or derivative thereof, is to be used to inhibit scarring associated with a fibrotic disorder, a therapeutically effective amount of the sFRP3, or fragment or derivative, may be provided by means of a number of administrations. Suitable regimes may involve administration monthly, weekly, daily or twice daily.

The inventors believe that therapeutically effective amounts of sFRP3, or its fragments or derivatives, may also be used to reduce existing scars. This is applicable to existing scars that result from the healing of a wound, and/or existing scars associated with fibrotic disorders. Accordingly the use of methods and medicaments of the invention in the reduction of existing scars constitutes a preferred use according to the invention. A therapeutically effective amount of sFRP3, or a fragment or derivative thereof, may be provided by means of any number of suitable administrations. Suitable regimes for these administrations may be readily devised by the skilled person using techniques (including in vitro studies, animal and human studies) well known in and established within the pharmaceutical industry.

It will be appreciated that the amount of a medicament of the invention that should be provided to a wound or fibrotic disorder, in order that a therapeutically effective amount of an active agent may be administered, depends on a number of factors.

These include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the active agent in the subject being treated.
B) The specific condition to be treated (e.g. acute wounding or chronic fibrotic disorders).
C) The age of the subject.
D) The size of the site to be treated.

The frequency of administration will also be influenced by the above-mentioned factors and particularly the half-life of the chosen agent within the subject being treated.

Generally when medicaments in accordance with the invention are used to treat existing scars (whether resulting from healing of a wound, or associated with a fibrotic disorder) the medicament should be administered as early as possible in the scarring process or as the fibrotic disorder begins. In the case of wounds or fibrotic disorders that are not immediately apparent, such as those at internal body sites, medicaments may be administered as soon as the wound or disorder, and hence the risk of scarring, is diagnosed. Therapy with methods or medicaments in accordance with the invention should continue until scarring has been inhibited to a clinician's satisfaction.

Frequency of administration will depend upon the biological half-life of the agent used. Typically a cream or ointment containing an agent of the invention should be administered to a target tissue such that the concentration of the agent at a wound or site of fibrosis is maintained at a level suitable to inhibit scarring. This may require administration daily or even several times daily. The inventors have found that administration of an agent of the invention immediately prior to wounding, with a further administration one day after wounding is particularly effective for the inhibition of scarring that would otherwise result from the healing of such a wound.

Daily doses of an agent of the invention may be given as a single administration (e.g. a daily application of a topical formulation or a daily injection). Alternatively, the agent of the invention may require administration twice or more times during a day. In a further alternative, a slow release device may be used to provide optimal doses of an agent of the invention to a patient without the need to administer repeated doses.

Routes of Administration

Therapeutically effective amounts of sFRP3, or of therapeutically effective fragments or derivatives thereof, may be administered by any suitable route capable of achieving the desired effect of inhibiting scarring. However, it may generally be preferred that sFRP3, or therapeutically effective fragments or derivatives thereof, are provided to a tissue, the scarring of which is to be inhibited, by local administration.

Suitable methods by which such local administration may be achieved will depend on the identity of the tissue or organ in question, and may also be influenced by whether the scarring to be inhibited is scarring resulting from the healing of a wound, or scarring associated with a fibrotic disorder. The selection of preferred routes of administration may also depend on whether or not a tissue or organ to be treated is permeable to the chosen medicament. Suitable routes of administration may be selected from the group consisting of: injections; application of sprays, ointments, or creams; inhalation of medicaments; release from biomaterials or other solid medicaments including sutures or wound dressings. Generally, preferred routes of administration may include local injection (for example intradermal injection in the case where it is wished to inhibit scarring of the skin). Suitable formulations for use in these embodiments of the invention are considered elsewhere in the specification.

Medicaments of the invention may be administered in a topical form to inhibit scarring (whether resulting from the healing of a wound, or associated with a fibrotic disorder). In the case of inhibiting scarring that would otherwise result from healing of a wound, such administration may be effected as part of the initial and/or follow up care for the wounded area. Injections may be administered around the margins of a wound, or a site of fibrosis. In the case of their prophylactic use, medicaments of the invention may be applied to a site where a wound or fibrotic disorder will occur.

Preferred routes of administration may be selected with reference to the tissue or organ to be treated. In the case of corneal scarring, medicaments of the invention may be administered to the outer surfaces of the eye, such as the cornea. Application of the medicament may be by means of local eye drops (including viscous or semi-viscous eye drops), creams, gels, ointments, or the like, and may, for example be applied using a sponge applicator.

In the case where it is wished to provide a therapeutically effective amount of sFRP3, or a fragment or derivative thereof, to internal wounds such as those caused by surgical procedures (which may otherwise be prone to formation of adhesions), medicaments may be administered by lavage, or in a parenteral gel/instillate or locally e.g. from sutures, films or carriers inserted at the time of surgery.

In the event that it is wished to inhibit scarring associated with fibrotic disorders such as proliferative vitreoretinopathy, it may be preferred to administer a therapeutically effective amount of an active agent by means of suitable injection (e.g. intravitreal injection) or by release from localised (e.g. intraocular) devices implanted in the eye. Suitable injections may preferably follow surgery or intravitreal implantation procedures.

In the case of scarring of blood vessels, suitable routes of administration may include direct injection into the walls of the blood vessel (for instance before suturing), bathing an anastomotic site in a medium comprising the sFRP3, fragment or derivative, or administration of the active agent by local applied devices, e.g. sutures or stents. Effective inhibition of scarring in blood vessels may be indicated by the maintenance of a normal level of blood flow following blood vessel injury.

Scarring associated with fibrotic disorders will frequently occur in relatively inaccessible tissues and organs, and it may be preferred that when scarring associated with a fibrotic disorder is to be inhibited sFRP3, or fragment or derivative thereof, be administered systemically. Suitable routes of administration include, without limitation, oral, transdermal, inhalation, parenteral, sublingual, rectal, vaginal and intranasal. By way of example, solid oral formulations (such as tablets or capsules) providing a therapeutically effective amount of sFRP3, or a fragment or derivative thereof, may be used for the inhibition of scarring associated with renal fibrosis or cirrhosis of the liver. Aerosol formulations for inhalation may be preferred as means for providing sFRP3, or therapeutically effective fragments or derivatives thereof, in the event that it is wished to inhibit scarring associated with chronic obstructive pulmonary disease or other fibrotic disorders of the lungs and airways.

It will be appreciated that many of the routes of administration described above may also be suitable for topical administration to a tissue in which it is wished to inhibit scarring (for example, inhalation or intranasal administration for inhibition of scarring in the respiratory system, whether as a result of the healing of a wound, or associated with a fibrotic disorder).

Preferred Formulations for Use in Accordance with the Invention

Generally, medicaments of the invention may be formulated and manufactured in any form that allows for the medicament to be administered to a patient such that a therapeutically effective amount of sFRP3, or a fragment or derivative thereof, is provided to a site where scarring is to be prevented, reduced or inhibited.

Medicaments of the invention may preferably be provided in the form of one of more dosage units providing a therapeutically effective amount (or a known fraction or multiple of a therapeutically effective amount) of sFRP3, or a fragment or derivative thereof. Methods of preparing such dosage units will be well known to the skilled person; for example see Remington's Pharmaceutical Sciences 18^{th} Ed. (1990).

Compositions or medicaments containing active agents may take a number of different forms depending, in particular, on the manner in which they are to be used. Thus, for example, they may be in the form of a liquid, ointment, cream, gel, hydrogel, powder or aerosol. All of such compositions are suitable for topical application to a site of scarring (for example, either a wound or a fibrotic disorder), and this represents a preferred means of administering agents of the invention to a subject (person or animal) in need of treatment.

The agents of the invention may be provided on a sterile dressing or patch, which may be used to cover a wound or fibrotic site where scarring is to be inhibited.

The agents of the invention may be released from a device or implant, or may be used to coat such a device e.g. a stent, or a controlled release device, or a wound dressing, or sutures for use in wound closure.

It will be appreciated that the vehicle of a composition comprising agents of the invention should be one that is well tolerated by the patient and allows release of the agent to the wound or fibrotic site. Such a vehicle is preferably biodegradeable, bioresolveable, bioresorbable and/or non-inflammatory.

If the composition is to be applied to an existing wound or fibrotic site, then the pharmaceutically acceptable vehicle will be one which is relatively “mild” i.e. a vehicle which is biocompatible, biodegradable, bioresolvable and non-inflammatory.

An agent of the invention, or a nucleic acid encoding such an agent (as considered further below), may be incorporated within a slow or delayed release device. Such devices may, for example, be placed on or inserted under the skin and the agent or nucleic acid may be released over days, weeks or even months.

Delayed release devices may be particularly useful for patients, such as those suffering from extensive or pathological scarring or from long-lasting scarring associated with a fibrotic disorder, who require long-term administration of therapeutically effective amounts of sFRP3, its fragments or derivatives. Such devices may be particularly advantageous when used for the administration of an agent or nucleic acid that would otherwise normally require frequent administration (e.g. at least daily administration by other routes).

A dose of a composition comprising an active agent may preferably be sufficient to provide a therapeutically effective amount of sFRP3, or a fragment or derivative thereof, in a single administration. However, it will be appreciated that each dose need not in itself provide a therapeutically effective amount of an active agent, but that a therapeutically effective amount may instead be built up through repeated administration of suitable doses.

Various suitable forms are known for compositions comprising agents of the invention. In one embodiment a pharmaceutical vehicle for administration of an active agent may be a liquid and a suitable pharmaceutical composition would be in the form of a solution. In another embodiment, the pharmaceutically acceptable vehicle is a solid and a suitable composition is in the form of a powder. In a further embodiment the active agent may be formulated as a part of a pharmaceutically acceptable trans-epidermal delivery system, e.g., a patch/dressing

A solid vehicle can include one or more substances that may also act as flavouring agents, lubricants, solubilizers, suspending agents, fillers, glidants, compression aids, binders or tablet-disintegrating agents; it can also comprise an encapsulating material. In powders, the vehicle is a finely divided solid that is in admixture with the finely divided agent of the invention. In tablets, the agent of the invention is mixed with a vehicle having the necessary compression properties in suitable proportions and compacted in the shape and size desired. The powders and tablets preferably contain up to 99% of the agent of the invention. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions. The active agent can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intradermal, intrastromal (cornea), intraadventitial (blood vessels) or subcutaneous injection. Sterile solutions can also be administered intravenously. The agent of the invention may be prepared as a sterile solid composition that may be dissolved or suspended at the time of administration using sterile water, saline, or other appropriate sterile injectable medium (such as PBS). Vehicles are intended to include necessary and inert binders, suspending agents, lubricants and preservatives.

In the situation in which it is desired to administer an agent of the invention by means of oral ingestion, it will be appreciated that the chosen agent will preferably be an agent having an elevated degree of resistance to degradation. For example, the active agent may be protected (using the techniques well known to those skilled in the art) so that its rate of degradation in the digestive tract is reduced.

Medicaments in accordance with the invention for use in the inhibition of scarring in the lungs or other respiratory tissues may be formulated for inhalation.

Medicaments in accordance with the invention for use in the inhibition of scarring in the body cavities e.g. abdomen or pelvis, may be formulated as a lavage, gel or instillate.

sFRP3, or a therapeutically effective fragment or derivative thereof, for use in the medicaments or methods of the invention, may be incorporated in a biomaterial, from which it may be released to inhibit scarring. Biomaterials incorporating active agents are suitable for use in many contexts, and at many body sites, where it is desired to inhibit scarring, but may be of particular utility in providing sFRP3, or a fragment or derivative thereof, to the eye (for example after retina surgery or glaucoma filtration surgery), or to sites where it is wished to inhibit restenosis or adhesions. The inventors believe that biomaterials incorporating active agents may be used in the manufacture of sutures, and such sutures represent a preferred embodiment of a medicament of the invention.

Known procedures, such as those conventionally employed by the pharmaceutical industry (e.g. in vivo experimentation, clinical trials etc), may be used to establish specific formulations of compositions comprising agents of the invention and precise therapeutic regimes for administration of such compositions (such as daily doses of the active agent and the frequency of administration).

Medicaments of the invention may be used to inhibit scarring as a monotherapy (e.g. through use of medicaments of the invention alone). Alternatively the methods or medicaments of the invention may be used in combination with other compounds or treatments for the inhibition of scarring. Suitable compounds that may be used as parts of such combination therapies will be well known to those skilled in the art.

Gene Therapy

The skilled person will appreciate that therapeutically effective amounts of sFRP3, or its fragments or derivatives, may be provided at sites where it is wished to inhibit scarring by virtue of cellular expression (commonly referred to as gene therapy). Such cellular expression must be controlled in order to prevent the accumulation of non-therapeutic amounts of such active agents, or even amounts that are capable of exacerbating scarring or fibrosis. Accordingly, the invention provides a method of inhibiting scar formation, the method comprising inducing cellular expression of a therapeutically effective amount of sFRP3, or a therapeutically effective fragment or derivative thereof, at a site where scarring is to be inhibited. Such a site may, for example be a wound, or a site of a fibrotic disorder.

Based on the teaching contained in the present specification, it will be a matter of routine experimentation for one skilled in the art to devise protocols by which cells may be induced to express therapeutically effective amounts of sFRP3 (or its fragments or derivatives).

For example, the skilled person will appreciate that such cellular expression of therapeutically effective amounts of sFRP3 may be achieved by manipulating naturally occurring expression of this molecule by cells in the region of the site to be treated.

Alternatively, and preferably, cells in the region of the site to be treated may be induced to express sFRP3, or therapeutically effective fragments or derivatives thereof, by means of the introduction of materials encoding such agents. Suitable materials may typically comprise nucleic acids such as DNA or RNA, and these may be devised based upon the sequences referred to in this specification.

Nucleic acids for use in this embodiment of the invention may be administered “as is”, for example by means of ballistic transfection, or as parts of a larger construct, which may be able to incorporate stably into cells so transfected. Suitable constructs may also contain regulatory elements, by which expression of a therapeutically effective amount of sFRP3, or a fragment or derivative thereof, may be achieved. Such constructs give rise to further aspects of the present invention.

Thus the invention also provides a construct encoding sFRP3, or a therapeutically effective fragment or derivative thereof, said construct being capable of expression, at a site where scarring is to be inhibited, to give rise to a therapeutically effective amount of the sFRP3, or therapeutically effective fragment or derivative. The invention also provides a method of inhibiting scarring, the method comprising administering a construct (as described above) to a site where scarring is to be inhibited such that a therapeutically effective amount of sFRP3, or a therapeutically effective fragment or derivative thereof, is expressed. The invention also provides the use of such a construct in the manufacture of a medicament for the inhibition of scarring.

It will be appreciated that many of the advantages that may be gained as a result of inhibiting scarring of humans are also applicable to other animals, particularly veterinary or domestic animals (e.g. horses, cattle, dogs, cats etc). Accordingly it will be recognised that the medicaments and methods of the invention may also be used inhibit scarring of non-human animals. Generally the same active agents that may be used to inhibit scarring of humans may also be used in such cases, however it may be preferred to use sFRP3 (or a therapeutically effective fragment or derivative thereof) that is derived from the same type of animal as is being treated (e.g. in the case of treatment of horses, use of equine sFRP3).

The invention will now be further described with reference to the accompanying Sequence Information, Experimental Results, and Figures, in which:

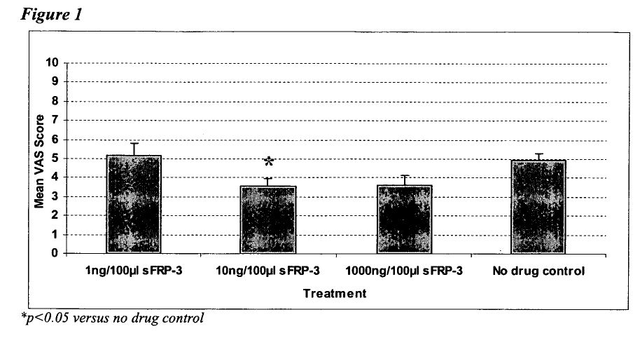

FIG. 1 is a bar graph and compares macroscopic Visual Analogue Scale (VAS) scores, indicative of the level of scarring in treated scars, with macroscopic VAS scores from scars produced on the healing of “no drug” naïve control wounds (* indicates p,0.05) all assessed 70 days after wounding.

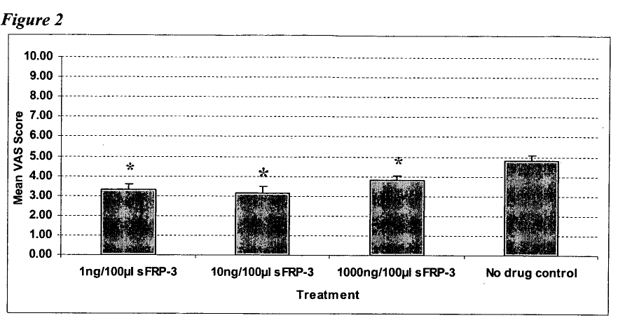

FIG. 2 is a bar graph and compares microscopic Visual Analogue Scale (VAS) scores, indicative of the level of scarring in treated scars, with microscopic VAS scores from scars produced on the healing of “no drug” naïve control wounds (* indicates p,0.05) all assessed 70 days after wounding.

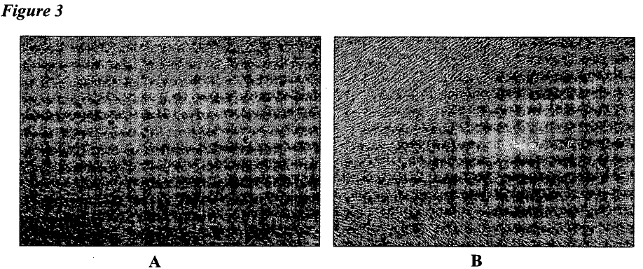

FIG. 3 compares representative images showing the macroscopic appearance of a treated scar (produced on healing of a wound treated with a total of 20 ng of sFRP3 by means of two administrations of a 10 ng/100 μl solution of sFRP3), on the left, with a scar produced on the healing of a “no drug” naïve control wound on the right.

Experimental Results

Carrier free secreted Frizzled Related Protein-3 (R&D Systems Cat. #192-SF/CF) was diluted in phosphate buffered saline (PBS) to produce medicaments of the invention having the following concentrations:

i) 1 ng/100 μL (a concentration of 0.26 nM, in which each 100 μl of the medicament provides 0.026 pmol of sFRP3);

ii) 10 ng/100 μL (a concentration of 2.6 nM, in which each 100 μl of the medicament provides 0.26 pmol of sFRP3); and

iii) 1000 ng/100 μL (a concentration of 263.0 nM, in which each 100 μl of the medicament provides 26.3 pmol of sFRP3).

Scarring Model

At Day 0, male Sprague Dawley rats (200-250g) were anaesthetised, shaved and wound sites were marked according to the Renovo rat incisional wounding template (2 wound model, 2×1 cm wounds at 5 cm from the base of the skull and 1 cm from the midline in each rat). One hundred microlitres of sFRP3 at ing, 10 ng or 1000 ng in phosphate buffered saline (PBS, pH 7.2; GIBCO BRL, Cat. #20012-019) was injected intradermally at the wound sites. The intradermal injections caused the formation of a raised bleb, which was then immediately incised to form 1 cm long experimental wounds. A separate group of rats were wounded without further treatment to act as naïve (no drug) controls. All treated wounds were re-injected again 1 day post-wounding with the appropriate treatment via injection of 50 μl to each of the two margins of the 1 cm wound and harvested at day 70 post-wounding.

Assessment of Scarring

The wounds were photographed after wounding, prior to re-injection on day 1 and on day of harvest. The scars were assessed using standard macroscopic scar assessment using a visual analogue scale (VAS) consisting of a 0-10 cm line representing a scale, from left to right, of 0 (corresponding to normal skin) to 10 (indicative of a bad scar).

For microscopic assessment the scars were excised from the experimental rats (incorporating a small amount of surrounding normal tissue) and fixed in 10% (v/v) buffered formal saline. The fixed tissue was then processed for wax histology, stained using Masson's trichrome, and scarring assessed using a microscopic visual analogue scale (VAS).

Results

Intradermal injections of sFRP3 at 10 ng/100 μl and 1000 ng/100 μl improved the macroscopic appearance of scars resulting from full thickness cutaneous incisional wounds, in comparison to scars resulting from “no drug” naive control wounds. The 10 ng/100 μl dose of sFRP3 resulted in a significant improvement in the macroscopic appearance of treated wounds (p<0.05).

Intradermal injections of sFRP3 at concentrations of 1 ng/100 μl, 10 ng/100 μl and 1000 ng/100 μl also significantly improved the microscopic appearance of scars resulting from full thickness cutaneous incisional wounds, in comparison to scars resulting from “no drug” naïve control wounds.

These results clearly illustrate the ability of therapeutically effective amounts of sFRP3, and hence of therapeutically effective fragments or derivatives of sFRP3, to prevent, reduce or inhibit scarring in vivo.

Sequence Information

| Sequence ID No. 1: | |
|---|---|
| 1 | mvcgspggml llragllala alcllrvpga raaacepvri plckslpwnm tkmpnhlhhs |
| 61 | tqanailaie qfegllgthc spdllfflca myapictidf qhepikpcks vcerarqgce |
| 121 | pilikyrhsw penlaceelp vydrgvcisp eaivtadgad fpmdssngnc rgasserckc |
| 181 | kpiratqkty frnnynyvir akvkeiktkc hdvtavvevk eilksslvni prdtvnlyts |
| 241 | sgclcpplnv neeyiimgye deersrlllv egsiaekwkd rlgkkvkrwd mklrhlglsk |
| 301 | sdssnsdstq sqksgrnsnp rgarn |
| Sequence ID No. 2: | |
| Amino acid sequence of sFRP-3 CRD (based on Dann et al., 2001) Conserved | |
| cysteine residues common to CRDs of Frizzled proteins are shown bold and | |
| underlined. | |
| 1 | aacepvripl ckslpwnmtk mpnhlhhstq anailaieqf egllgthcsp dllfflcamy |
| 61 | apictidfqh epikpcksvc erarqgcepi likyrhswpe nlaceelpvy drgvcispea |
| 121 | ivtad |
| Sequence ID No. 3: | |
| 1 | gttgggaaag agcagcctgg gcggcagggg cggtggctgg agctcggtaa agctcgtggg |
| 61 | accccattgg gggaatttga tccaaggaag cggtgattgc cgggggagga gaagctccca |
| 121 | gatccttgtg tccacttgca gcgggggagg cggagacggc ggagcgggcc ttttggcgtc |
| 181 | cactgcgcgg ctgcaccctg ccccatcctg ccgggatcat ggtctgcggc agcccgggag |
| 241 | ggatgctgct gctgcgggcc gggctgcttg ccctggctgc tctctgcctg ctccgggtgc |
| 301 | ccggggctcg ggctgcagcc tgtgagcccg tccgcatccc cctgtgcaag tccctgccct |
| 361 | ggaacatgac taagatgccc aaccacctgc accacagcac tcaggccaac gccatcctgg |
| 421 | ccatcgagca gttcgaaggt ctgctgggca cccactgcag ccccgatctg ctcttcttcc |
| 481 | tctgtgccat gtacgcgccc atctgcacca ttgacttcca gcacgagccc atcaagccct |
| 541 | gtaagtctgt gtgcgagcgg gcccggcagg gctgtgagcc catactcatc aagtaccgcc |
| 601 | actcgtggcc ggagaacctg gcctgcgagg agctgccagt gtacgacagg ggcgtgtgca |
| 661 | tctctcccga ggccatcgtt actgcggacg gagctgattt tcctatggat tctagtaacg |
| 721 | gaaactgtag aggggcaagc agtgaacgct gtaaatgtaa gcctattaga gctacacaga |
| 781 | agacctattt ccggaacaat tacaactatg tcattcgggc taaagttaaa gagataaaga |
| 841 | ctaagtgcca tgatgtgact gcagtagtgg aggtgaagga gattctaaag tcctctctgg |
| 901 | taaacattcc acgggacact gtcaacctct ataccagctc tggctgcctc tgccctccac |
| 961 | ttaatgttaa tgaggaatat atcatcatgg gctatgaaga tgaggaacgt tccagattac |
| 1021 | tcttggtgga aggctctata gctgagaagt ggaaggatcg actcggtaaa aaagttaagc |
| 1081 | gctgggatat gaagcttcgt catcttggac tcagtaaaag tgattctagc aatagtgatt |
| 1141 | ccactcagag tcagaagtct ggcaggaact cgaacccccg gcaagcacgc aactaaatcc |
| 1201 | cgaaatacaa aaagtaacac agtggacttc ctattaagac ttacttgcat tgctggacta |
| 1261 | gcaaaggaaa attgcactat tgcacatcat attctattgt ttactataaa aatcatgtga |
| 1321 | taactgatta ttacttctgt ttctcttttg gtttctgctt ctctcttctc tcaacccctt |
| 1381 | tgtaatggtt tgggggcaga ctcttaagta tattgtgagt tttctatttc actaatcatg |
| 1441 | agaaaaactg ttcttttgca ataataataa attaaacatg ctgttaccag agcctctttg |
| 1501 | ctggagtctc cagatgttaa tttactttct gcaccccaat tgggaatgca atattggatg |
| 1561 | aaaagagagg tttctggtat tcacagaaag ctagatatgc cttaaaacat actctgccga |
| 1621 | tctaattaca gccttatttt tgtatgcctt ttgggcattc tcctcatgct tagaaagttc |
| 1681 | caaatgttta taaaggtaaa atggcagttt gaagtcaaat gtcacatagg caaagcaatc |
| 1741 | aagcaccagg aagtgtttat gaggaaacaa cacccaagat gaattatttt tgagactgtc |
| 1801 | aggaagtaaa ataaatagga gcttaagaaa gaacattttg cctgattgag aagcacaact |
| 1861 | gaaaccagta gccgctgggg tgttaatggt agcattcttc ttttggcaat acatttgatt |
| 1921 | tgttcatgaa tatattaatc agcattagag aaatgaatta taactagaca tctgctgtta |
| 1981 | tcaccatagt tttgtttaat ttgcttcctt ttaaataaac ccattggtga aagtcccaaa |
| 2041 | aaaaaaaaaa aaaaaaaa |

1-16. (canceled)

17. A method of preventing, reducing or inhibiting scarring, the method comprising administering a therapeutically effective amount of sFRP3, or a therapeutically effective fragment or derivative thereof, to a patient in need of such prevention, reduction or inhibition.

18. A method according to claim 17, wherein the sFRP3 is administered as a topical medicament for application at a site where scarring is to be prevented, reduced or inhibited.

19. A method according to claim 17, wherein the sFRP3 is administered by localised injection.

20. A method according to claim 17, wherein the scarring prevented, reduced or inhibited is scarring of the skin.

21. A method according to claim 17, wherein the scarring prevented, reduced or inhibited is scarring associated with a wound.

22. A method according to claim 17, wherein the scarring prevented, reduced or inhibited is scarring associated with a wound selected from the group consisting of: wounds of the skin; wounds of the eye, including eye wounds resulting from eye surgery such as LASIK surgery, LASEK surgery, PRK surgery, or cataract surgery; wounds subject to capsular contraction; wounds of blood vessels; wounds of the central and peripheral nervous system; wounds of tendons, ligaments or muscle; wounds of the oral cavity, including wounds of the lips and palate; wounds of the internal organs including wounds of the liver, heart, brain, digestive tissues and reproductive tissues; and wounds of body cavities, including wounds of the abdominal cavity, pelvic cavity and thoracic cavity.

23. A method according to claim 17, wherein the scarring prevented, reduced or inhibited is scarring associated with a surgical wound.

24. A method according to claim 17, wherein the scarring prevented, reduced or inhibited is scarring associated with a fibrotic disorder.

25. A method according to claim 17, wherein the scarring prevented, reduced or inhibited is scarring associated with a fibrotic disorder selected from the group consisting of:
skin fibrosis; scleroderma; conjunctival cicatrisation; progressive systemic fibrosis; lung fibrosis;
muscle fibrosis; kidney fibrosis; glomerulosclerosis; glomerulonephritis; uterine fibrosis; renal fibrosis; cirrhosis of the liver, liver fibrosis; chronic obstructive pulmonary disease; fibrosis following myocardial infarction; central nervous system fibrosis, such as fibrosis following stroke; fibrosis associated with neuro-degenerative disorders such multiple sclerosis; fibrosis associated with proliferative vitreoretinopathy (PVR); restenosis; endometriosis; ischemic disease and radiation fibrosis.

26. A method according to claim 17, wherein the sFRP3, fragment or derivative is administered in an amount of between about 2.6fmol and 40 pmol, per centimetre of wound or centimetre of fibrosis.

27. A method according to claim 17, wherein sFRP3 is administered to the patient.

28. A method according to claim 17, a therapeutically effective fragment of sFRP3, selected from the group consisting of: a fragment comprising the CRD of sFRP3; a fragment comprising the C-terminal domain of sFRP3; a fragment comprising the pharmacophore of sFRP3; and a glycosylated fragment of sFRP3, is administered to the patient.

29. A method according to claim 17, a therapeutically effective derivative of sFRP3 selected from the group consisting of: therapeutically effective derivatives based on the pharmacophore of sFRP3; therapeutically effective peptoid derivatives of sFRP3 or its fragments; therapeutically effective D-amino acid derivatives of sFRP3 or its fragments; therapeutically effective peptidomimetics based on sFRP3 or its fragments; therapeutically effective peptide analogues of sFRP3 or its fragments; therapeutically effective pseudopeptides based on sFRP3 or its fragments; therapeutically effective retro-inverso peptides based on sFRP3 or its fragments; therapeutically effective depsipeptide derivatives based on sFRP3 or its fragments; therapeutically effective β-peptide derivatives based on sFRP3 or its fragments; therapeutically effective small molecule mimics of sFRP3; and therapeutically effective retropeptoid derivatives based on sFRP3 or its fragments, is administered to the patient.
